(19) United States
(12) Patent Application Publication
(10) Pub. No.: US 2011/0125144 A1
(43) Pub. Date: May 26, 2011

(54) METHOD OF TREATING ATRIAL FIBRILLATION THROUGH EPICARDIAL LESION

(75) Inventor: James R. Edgerton, Plano, TX (US)

(73) Assignee: BAYLOR RESEARCH INSTITUTE, Dallas, TX (US)

(21) Appl. No.: 12/625,696

(22) Filed: Nov. 25, 2009

(51) Int. Cl. A61B 18/18

(52) U.S. Cl. 606/33

(57) ABSTRACT

A method of treating atrial fibrillation through minimal access ablation to form atrial lesions on the dome of the atrium.

### TECHNICAL FIELD OF THE INVENTION

The present invention relates in general to the field of treating atrial fibrillation, specifically to improved method for treating atrial fibrillation using ablation.

### STATEMENT OF FEDERALLY FUNDED RESEARCH

None.

### CROSS-REFERENCE TO RELATED APPLICATIONS

None.

### INCORPORATION-BY-REFERENCE OF MATERIALS FILED ON COMPACT DISC

None.

### BACKGROUND OF THE INVENTION

Without limiting the scope of the invention, its background is described in connection with improved method for treating atrial fibrillation using ablation.

Atrial fibrillation is one of the most common heart arrhythmias, affecting millions of patients in the U.S. alone. It is a rapid, irregular heart rhythm originating in the atrial chambers of the heart, commonly causing palpitations and fatigue and greatly increases the risk of stroke. There have been many approaches to treating atrial fibrillation.

One example is ablation, in which the source of a patient's heart arrhythmia is mapped, localized, and then destroyed. Generally, ablation is accomplished by applying radiofrequency (RF) energy, applying electrical energy, or freezing the offending area, thus creating a small scar that is electrically inactive and thus incapable of generating heart arrhythmias. Many forms of cardiac arrhythmias have been rendered curable by ablation techniques, but atrial fibrillation has remained a challenge since the electrical abnormalities associated with atrial fibrillation have been thought to be much more generalized—essentially encompassing most of the left and right atrium.

For example, U.S. Pat. No. 7,588,568, entitled, “Atrial ablation catheter and method for treating atrial fibrillation” that discloses a catheter for ablating tissue is provided. The catheter comprises an elongated generally-tubular catheter body having proximal and distal ends and at least one lumen extending therethrough. A non-retractable ablation assembly is attached to the distal end of the catheter body. The ablation assembly comprises proximal and distal non-conductive tubings, each having a lumen extending therethrough and a generally tubular electrode mounted between the proximal and distal non-conductive tubings. The tubular electrode is formed of a material having shape-memory and has at least one irrigation port through which fluid can pass from the inside to the outside of the electrode. The ablation assembly further comprises a non-conductive protective tubing extending generally parallel to and along the outside of the tubular electrode. The protective tubing has proximal and distal ends extending into the proximal and distal non-conductive tubings, respectively. The catheter further comprises at least one of an electrode lead wire and a temperature sensor wire, and preferably both, extending through the non-conductive protective tubing and catheter body, the electrode lead wire having a distal end mounted to a ring electrode mounted on the distal non-conductive tubing, and the temperature sensor wire having a distal end mounted on or under the distal non-conductive tubing. The catheter also comprises an infusion tube extending through the catheter body and having a distal end in fluid communication with the proximal end of the tubular electrode.

### SUMMARY OF THE INVENTION

Improvements in enabling technology have facilitated minimal access techniques to the surgical ablation of atrial fibrillation (AF). A variety of lesion sets (usually targeting only the left atrium) have been utilized in attempts to ablate AF. However, it has heretofore not been possible to perform the full Cox Maze III left atrial lesion set with minimal access techniques on the full beating heart. We describe a new epicardial approach to apply a set of left atrial lesions which are electrophysiologically equivalent to all the left atrial lesions of the Cox Maze III while using minimal access techniques.

The present invention provides a method of forming atrial lesions on the dome of the atrium by positioning the patient on a table; placing an object under a thorax area of the patient to raise the thorax 3 to 18 inches to gain access to a posterolateral thorax area; preparing access to a lateral thorax area bilaterally and a sternum area of the patient; placing one or more external defibrillator pads on the patient; inserting a first right port in a mid-axillary line in the third intercostal space of the patient; insufflating the patient with CO₂ to expand the field and depresses the diaphragm; inserting a second right port in a mid-clavicular line in approximately a second intercostal space of the patient; inserting a third right port in a mid-axillary line in approximately the 7^{th} intercostal space of the patient, wherein the second right port and third right port are positioned most cephalad and the other is positioned most caudad so that an instrument can access the transverse sinus, behind the superior vena cava and immediately cephalad to the right atrial appendage; sealing at least partially the first right port, the right second port and the third right port; dissecting the fibroareolar tissue between the inferior vena cava and the right inferior pulmonary vein to form an opening into the posterior pericardium; converting a portion of the superior vena cava to an intrapericardial area; dividing the tissue between the superior vena cava and the right pulmonary artery; dissecting the fat pad located behind the superior vena cava and in front of the left atrial muscular dome to expose the atrium muscular dome; removing the second right port or the third right port and insert a lighted dissector; advancing the lighted dissector between the right inferior pulmonary vein and the inferior vena cava into the posterior pericardium; dissecting through the space between the right pulmonary artery and the right superior pulmonary vein with the lighted dissector; removing the lighted dissector; inserting a sensing pen through the most cephalad port; obtaining a right baseline electrogram on multiple sites on both the superior and inferior pulmonary veins with the sensing pen; introducing a closed bipolar clamp comprising a jaw and a posterior jaw a through the most caudad port site; positioning the posterior jaw of the clamp behind the right-sided pulmonary veins until the jaw and the posterior jaw are well-up on the antrum of the pulmonary vein and well-away from the bifurcation of the pulmonary veins; producing an ablation line on the right pulmonary vein antrum by firing the clamp 2-6 times; obtaining a second right electrogram on multiple sites on both the superior and inferior pulmonary veins with the sensing pen, wherein the absence of transmitted electrical activity from the atrium indicates acute entrance block has been obtained; introducing a linear bipolar radiofrequency device through the most caudad port; positioning the linear bipolar ablation device behind the superior vena cava; forming a first linear ablation line from the right superior pulmonary vein across the dome of the left atrium pointing towards the left superior pulmonary vein; positioning the linear bipolar ablation device at the junction of the noncoronary cusp and the left coronary cusp of the aorta; forming a second linear ablation line from the fibrous trigone obliquely on the dome to connect the left fibrous trigone to the transverse ablation line across the dome of the atrium; closing the pericardium; withdrawing the first right port, the second right port and the third right port; closing a first right port incision, a second right port incision and a third right port incision; inflating the lung; positioning a first left port in a mid-axillary line in the third intercostal space of the patient; insufflating the patient with CO2 to expand the field and depresses the diaphragm; positioning a second left port in a mid-clavicular line in approximately a second intercostal space of the patient; positioning a third left port in a mid-axillary line in approximately the 6^{th} or 7^{th} intercostal space of the patient, wherein the second left port and third left port are positioned so that an instrument can access the transverse sinus, behind the superior vena cava and immediately cephalad to the right atrial appendage; sealing at least partially the first left port, the second left port and the third left port; opening the pericardium posterior to the phrenic nerve; inserting the sensing pen through the most cephalad port; obtaining a left baseline electrogram in the pulmonary veins with the sensing pen; dividing the ligament of Marshall posterior; introducing the lighted dissector having a tip into the most caudad port; directing the lighted dissector around the pulmonary veins with the tip positioned at the point of the divided ligament of Marshall; introducing a closed bipolar clamp having a jaw and a posterior jaw through the most-caudad port site; positioning the posterior jaw behind the pulmonary vein and the clamp is closed well up on the pulmonary vein antrum firing the bipolar clamp 3 times changing the position of the clamp each time; removing the lighted dissector; obtaining a second left electrogram in the pulmonary veins wherein electrical silence indicates entrance block so that no atrial electrical activity is transmitted into the veins; introducing the linear bipolar radiofrequency device through the most caudad port site; forming a third linear ablation line that connects the right superior pulmonary vein over to the left superior pulmonary vein as far posteriorly as can be done in a transverse sinus; forming a forth linear ablation line from the left fibrous trigone to the right superior pulmonary vein; wherein the first linear ablation line, the second linear ablation line, the third linear ablation line, and the fourth linear ablation line form an inverted triangle on the dome; obtaining a triangle electrogram of the activity within the inverted triangle wherein a flat electrogram indicates no conducted atrial activity; closing the pericardium; withdrawing the first left port, the second left port and the third left port; closing a first left port incision, a second left port incision and a third left port incision; and inflating the lung.

The present invention provides a method of forming atrial lesions on the dome of the atrium by positioning the patient on a table; placing 3 to 6 blankets under a thorax area of the patient to raise the to gain access to a posterolateral thorax area; preparing access to a lateral thorax area bilaterally and a sternum area of the patient; placing one or more external defibrillator pads on the patient; positioning a spinal needle through the chest wall with the tip targeting the transverse sinus to determine the exact placement of a first 5 mm right port, a second 10 mm right port or a 10 mm right third port; inserting the first 5 mm right port in a mid-axillary line in the third intercostal space of the patient; insufflating the patient with CO₂ to expand the field and depresses the diaphragm; inserting a second right port in a mid-clavicular line in approximately a second intercostal space of the patient; inserting a third right port in a mid-axillary line in approximately the 7^{th} intercostal space of the patient, wherein the second right port or third right port is positioned most cephalad and the other is positioned most caudad so that an instrument can access the transverse sinus, behind the superior vena cava and immediately cephalad to the right atrial appendage; sealing at least partially the first right port, the right second port and the third right port; dissecting the fibroareolar tissue between the inferior vena cava and the right inferior pulmonary vein to form an opening into the posterior pericardium; converting a portion of the superior vena cava to an intrapericardial area; dividing the tissue between the superior vena cava and the right pulmonary artery; dissecting the fat pad located behind the superior vena cava and in front of the left atrial muscular dome to expose the atrium muscular dome; removing the second right port or the third right port and insert a lighted dissector; advancing the lighted dissector between the right inferior pulmonary vein and the inferior vena cava into the posterior pericardium; dissecting through the space between the right pulmonary artery and the right superior pulmonary vein with the lighted dissector; removing the lighted dissector; inserting a sensing pen through the most cephalad port; obtaining a right baseline electrogram on multiple sites on both the superior and inferior pulmonary veins with the sensing pen; introducing a closed bipolar clamp comprising a jaw and a posterior jaw a through the most caudad port site; positioning the posterior jaw of the clamp behind the right-sided pulmonary veins until the jaw and the posterior jaw are well-up on the antrum of the pulmonary vein and well-away from the bifurcation of the pulmonary veins; producing an ablation line on the right pulmonary vein antrum by firing the clamp 2-6 times; obtaining a second right electrogram on multiple sites on both the superior and inferior pulmonary veins with the sensing pen, wherein the absence of transmitted electrical activity from the atrium indicates acute entrance block has been obtained; introducing a linear bipolar radiofrequency device through the most caudad port; positioning the linear bipolar ablation device behind the superior vena cava; forming a first linear ablation line from the right superior pulmonary vein across the dome of the left atrium pointing towards the left superior pulmonary vein; positioning the linear bipolar ablation device at the junction of the noncoronary cusp and the left coronary cusp of the aorta; forming a second linear ablation line from the fibrous trigone obliquely on the dome to connect the left fibrous trigone to the transverse ablation line across the dome of the atrium; closing the pericardium; withdrawing the first 5 mm right port, the second 10 mm right port and the third 10 mm right port; closing a first right port incision, a second right port incision and a third right port incision; inflating the lung; positioning a first 5 mm left port in a mid-axillary line in the third intercostal space of the patient; insufflating the patient with CO2 to expand the field and depresses the diaphragm; positioning a second 10 mm left port in a mid-clavicular line in approximately a second intercostal space of the patient; positioning a third 10 mm left port in a mid-axillary line in approximately the 6^{th} or 7^{th} intercostal space of the patient, wherein the second 10 mm left port and third 10 mm left port are positioned so that an instrument can access the transverse sinus, behind the superior vena cava and immediately cephalad to the right atrial appendage; sealing at least partially the first left port, the second left port and the third left port; opening the pericardium posterior to the phrenic nerve; inserting the sensing pen through the most cephalad port; obtaining a left baseline electrogram in the pulmonary veins with the sensing pen; dividing the ligament of Marshall posterior; introducing the lighted dissector having a tip into the most caudad port; directing the lighted dissector around the pulmonary veins with the tip positioned at the point of the divided ligament of Marshall; introducing a closed bipolar clamp having a jaw and a posterior jaw through the most-caudad port site; positioning the posterior jaw behind the pulmonary vein and the clamp is closed well up on the pulmonary vein antrum; firing the bipolar clamp 3 times changing the position of the clamp each time; removing the lighted dissector; obtaining a second left electrogram in the pulmonary veins wherein electrical silence indicates entrance block so that no atrial electrical activity is transmitted into the veins; introducing the linear bipolar radiofrequency device through the most caudad port site; forming a third linear ablation line that connects the right superior pulmonary vein over to the left superior pulmonary vein as far posteriorly as can be done in a transverse sinus; forming a forth linear ablation line from the left fibrous trigone to the right superior pulmonary vein; wherein the first linear ablation line, the second linear ablation line, the third linear ablation line, and the fourth linear ablation line form an inverted triangle on the dome; obtaining a triangle electrogram of the activity within the inverted triangle wherein a flat electrogram indicates no conducted atrial activity; closing the pericardium; withdrawing the first 5 mm left port, the second 10 mm left port and the third 10 mm left port; closing a first 5 mm left port incision, a second 10 mm left port incision and a third 10 mm left port incision; and inflating the lung.

### BRIEF DESCRIPTION OF THE DRAWINGS

For a more complete understanding of the features and advantages of the present invention, reference is now made to the detailed description of the invention along with the accompanying figures and in which:

FIG. 1 is an image of the heart illustrating that by working behind the superior vena cava and through the transverse sinus, a transverse connecting lesion can be placed across the dome of the left atrium connecting the right superior PV with the left superior PV (roof line) and a connecting lesion can be made from the roof line to the anterior mitral valve at the left fibrous trigone. The epicardial landmark for connection to the mitral valve is the junction of the aortic left cusp and the noncoronary cusp in the transverse sinus.

FIG. 2 is an image of the heart illustrating the surgical approach to the transverse sinus.

FIG. 3A is an image of the body illustrating the proper positioning of the patient in the operating room which is critical in order to obtain a successful surgical result.

FIG. 3B AND FIG. 3C are images of the heart three ports positioned on the patient illustrating that the surgeon must have free access to the posterior axillary line bilaterally.

FIG. 4A is an image of the heart illustrating that by working through these port sites, the right pericardium is open from the diaphragm to the level of the aorta approximately 2 cm anterior to the phrenic nerve.

FIG. 4B is an image of the heart illustrating that the pericardium is bluntly and sharply dissected away until a portion of the superior vena cava is converted to intrapericardial.

FIG. 5 is an image of the heart illustrating that the lighted dissector will not fit through the 10 mm port, but will easily slide through the port site.

FIG. 6 is an image of the heart illustrating that a sensing pen is passed through the most cephalad port site and used to obtain a baseline electrogram on multiple sites on both the superior and inferior pulmonary veins.

FIG. 7 is an image of the heart illustrating the internally cooled, linear bipolar radiofrequency device introduced through the most caudad port site.

FIG. 8 is an image of the heart illustrating the tip of the ablation device articulated to the right and further bending of the malleable shaft with the tip positioned at the junction of the noncoronary cusp and the left coronary cusp of the aorta.

FIG. 9 is an image of the heart three ports positioned on the left side of the patient that is similar to the right side, but somewhat more posterior.

FIG. 10 is an image of the heart illustrating that the pericardium is posterior to the phrenic nerve.

FIG. 11 is an image of the heart illustrating the transverse sinus and the lesions placed from the right side.

FIG. 12 is an image of the heart illustrating the bipolar radiofrequency clamp introduced through the most caudad port site.

FIG. 13 is an image of the heart illustrating that by The clamp is withdrawn and the linear radiofrequency ablation device is introduced through the most caudad port site.

FIG. 14 is an image of the heart illustrating the ablations that have been done on the dome and an inverted triangle constructed.

FIG. 15 is an image of the heart illustrating a stapling device introduced through the most caudad port site and positioned around the base of the atrial appendage.

FIG. 16 is a schematic of the location of the connecting lesions on the dome of the atrium and position of the temporary pacing/recording electrodes.

FIG. 17A is a schematic of the electrode placement to verify activation block across the transverse dome line. FIG. 17B is a schematic indicating the electrode placement to confirm complete block across the left fibrous trigone line.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Minimal access ablation of atrial fibrillation has undergone a progression. For Example, the lesion set has progressed from simple pulmonary vein isolation to a more comprehensive lesion set which can be placed epicardially, and more closely replicates the left atrial lesions of the Cox Maze III. Access has progressed from bilateral mini thoracotomies initially described by Wolf [1], to a totally thorascopic approach highly modified from that initially described by Puskas [2] and Van Boven [3]. The present invention provides, seventy-four patients (46 paroxysmal, 14 persistent, 14 longstanding persistent) that underwent bilateral PV antral electrical isolation using a bipolar radiofrequency clamp. The groups were defined as follows: paroxysmal was recurrent atrial fibrillation that terminates spontaneously within 7 days; persistent atrial fibrillation was defined as atrial fibrillation sustained beyond seven days, or lasting less than seven days but requiring either pharmacologic or electrical cardioversion; longstanding persistent atrial fibrillation was continuous atrial fibrillation of greater than one-year duration. Of the 74 patients, 13 had undergone one prior ablation, six had two prior ablations, and one patient had three prior ablations. Fifteen of the 74 had permanent pacemakers in place. All patients underwent TEEs on the table prior to the procedure, and those who had undergone a prior catheter ablation had a left atrial MRI or left atrial CT scan.

Rhythm was monitored by office EKG at one, three, and six months. At six months, the burden of atrial fibrillation was assessed by a 14 to 21-day auto-triggered event monitor. When patient circumstances dictated, a 24-hour Holter monitor was substituted for the 21 day event monitor. The long term monitors used samples the rhythm at 15-second intervals. When atrial fibrillation was detected by irregular rhythm, a single 15-second rhythm strip was recorded. Another rhythm strip was not recorded during that episode. Accordingly, we counted the number of episodes of atrial fibrillation but were unable to determine the duration of episodes over 15-seconds or the true burden of atrial fibrillation. Patients with pacemakers underwent pacemaker interrogation for episodes of atrial fibrillation. (Table 1)

TABLE 1

| | Paroxysmal | | Persistent/Longstanding Persistent | |
|---|---|---|---|---|
| | Atrial Fibrillation Patients | | Atrial Fibrillation Patients | |
| | N = 46 | | N = 28 | |
| | | Holter/PM Interrogation/ | | Holter/PM Interrogation/ |
| | EKG | Event Monitor | EKG | Event Monitor |
| | N = 43 | N = 43 | N = 27 | N = 23 |
| Follow Up: | NSR | NSR | NSR | NSR |
| 6 Months | 43 | 36 | 22 | 13 |
| | (100.0%) | (83.7%) | (81.5%) | (56.5%) |
| 6 Months | 33 | 30 | 13 | 8 |
| off AAD | (76.7%) | (69.8%) | (48.1%) | (34.8%) |

One might expect that PV antral isolation and partial autonomic denervation would not be adequate treatment for patients in persistent and long standing persistent atrial fibrillation because of the associated changes in the left atrial substrate which occur in these conditions [4]. This electrical remodeling of the left atrium results in a fixed shortened refractory period and a shortened fibrillatory interval [5]. Passive mechanical stretch of the left atrium that occurs in this chronic condition can in itself be arrythmogenic [6, 7]. Atrial fibrosis has also been associated with atrial fibrillation [8, 9]. Therefore, it is likely that isolating the PV triggers alone is insufficient treatment for persistent and long standing persistent atrial fibrillation. Hence, a more extensive lesion set similar to the left sided Cox Maze III is necessary.

To accomplish a lesion set replicating the left-sided Cox Maze III lesion set, a connecting lesion would need to be added between the left and right-sided PVs, a connecting lesion to the base of the atrial appendage, and a connecting lesion to the mitral valve annulus. Traditionally, these connecting lesions are placed endocardially between the left and right inferior PVs, and from the right inferior PV down to the mitral valve annulus crossing the left atrial isthmus. However, when the epicardium is approached in a minimally invasive fashion, there are three inhibitors to placing the lesions in these locations. First, with currently available techniques, there is little to no visualization affordable posteriorly behind the left atrium. Second, as the circumflex coronary artery frequently overlies the mitral valve annulus, there is significant risk for collateral damage to this vital structure. Finally, the epicardial landmark for the mitral annulus is the coronary sinus. However, it has been shown [10] that the coronary sinus can be up to 13 mm away from the mitral valve annulus, thus risking an incomplete connection which may result in left atrial flutter.

However, in the minimally invasive approach, we can obtain excellent visualization through the transverse sinus behind the aorta and the pulmonary artery. Therefore, we have conceived of and developed a lesion set (the Dallas Lesion Set) that places all of the connecting lesions on the dome of the atrium.

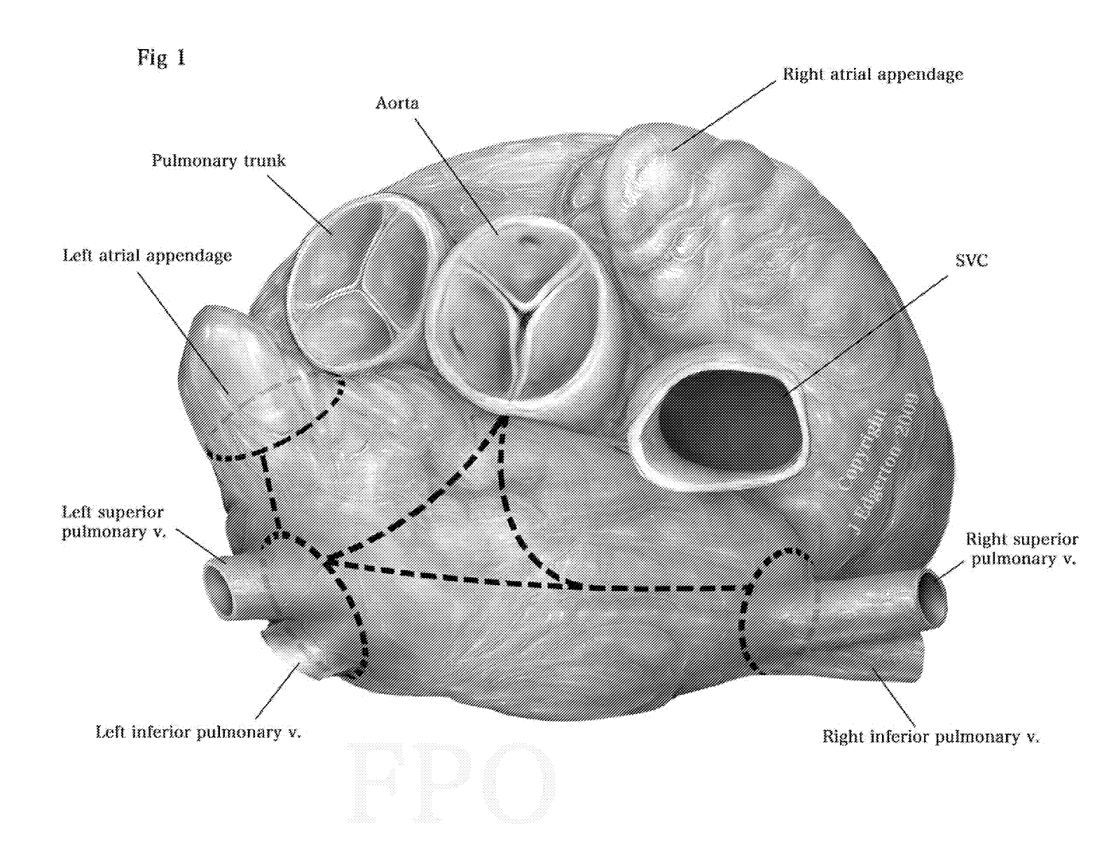

FIG. 1 is an image of the heart illustrating that by working behind the superior vena cava and through the transverse sinus, a transverse connecting lesion can be placed across the dome of the left atrium connecting the right superior PV with the left superior PV. By working behind the superior vena cava and through the transverse sinus, the present invention allows the transverse connecting lesion across the dome of the left atrium connecting the right superior PV with the left superior PV. It is then only a short extension of this line on the left side that connects it to the base of the left atrial appendage. The connecting lesion to the mitral valve annulus can also be accomplished on the dome of the left atrium within the transverse sinus. The left fibrous trigone touches the mitral valve annulus and connects it to the aorta at the aortic root. The left fibrous trigone meets the aortic valve at a point where the left coronary cusp and the non-coronary cusp join. Therefore, with good visualization, we can place this connecting lesion from the left fibrous trigone at the anterior mitral valve annulus, across the anterior dome of the atrium and touching transverse dome line. This then replicates all of the left atrial lesions of the classic Cox Maze III operation. Another short linear lesion connecting the left superior pulmonary vein to the left fibrous trigone then constructs a closed triangle on the dome of the atrium. By sensing and pacing within this triangle, one can test for entrance and exit block, thus demonstrating acute conduction block of all the dome lesions.

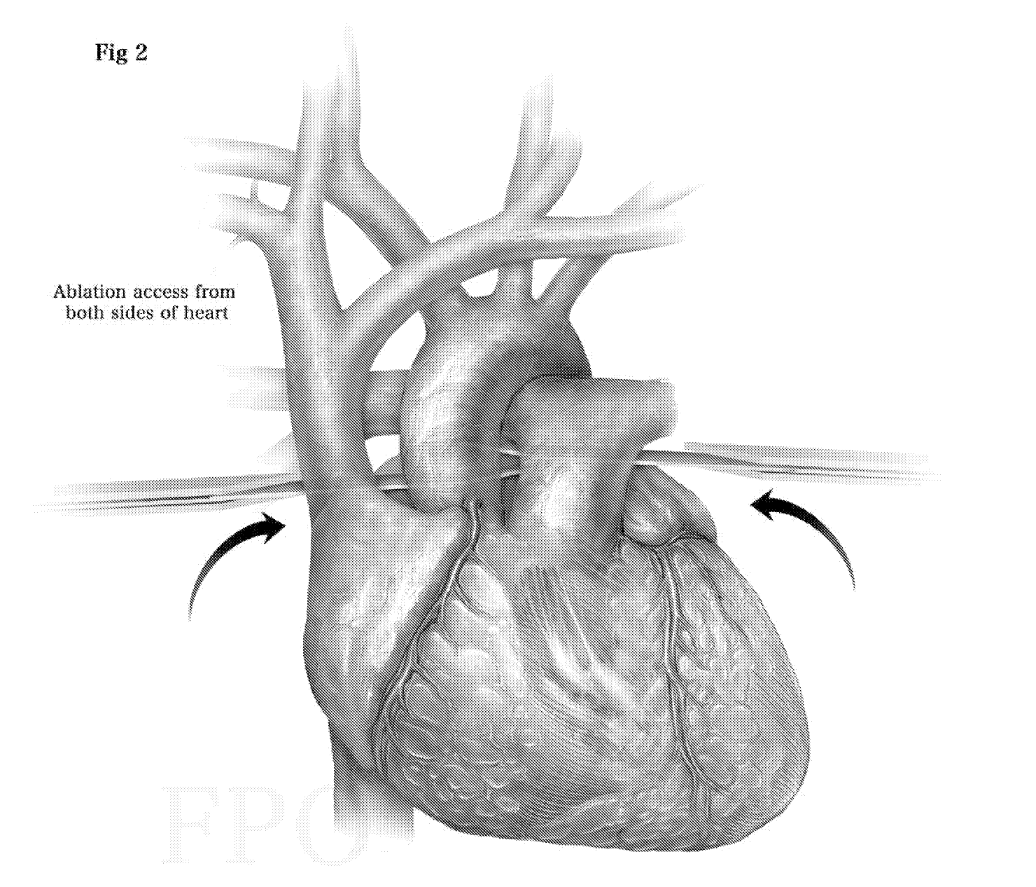

FIG. 2 shows the surgical approach to the transverse sinus which is further detailed in the specification. Twenty-nine patients (e.g., 10 persistent, 19 long standing persistent) were operated on using the Dallas Extended Lesion Set of the present invention with intra-operative mapping for confirmation of block. The median left atrial size was 5.4 cm. Follow-up at 6 months was with 2 week event monitor (20/29), pacemaker interrogation (8/29), or ECG (1/29). At 6 months 23/29 (79.3%) were free of atrial fibrillation. The patient who had only ECG follow-up was in AF on ECG and refused a longer term monitor.

Only one of the patients failed as persistent atrial fibrillation. Those who failed all failed as paroxysmal with a low residual burden of atrial fibrillation and were asymptomatic. Of the 6 failures, two patients had 1 episode, one patient had 2 episodes, one patient had 4 episodes, one patient had 6 episodes and one had ECG alone for an undetermined number of episodes. All of these were “symptomatic successes”. The other failure had >50 episodes of continuous atrial fibrillation. Results with a minimally invasive extended linear lesion set of the present invention suggest increased efficacy over pulmonary vein isolation for patients with persistent and long standing persistent atrial fibrillation.

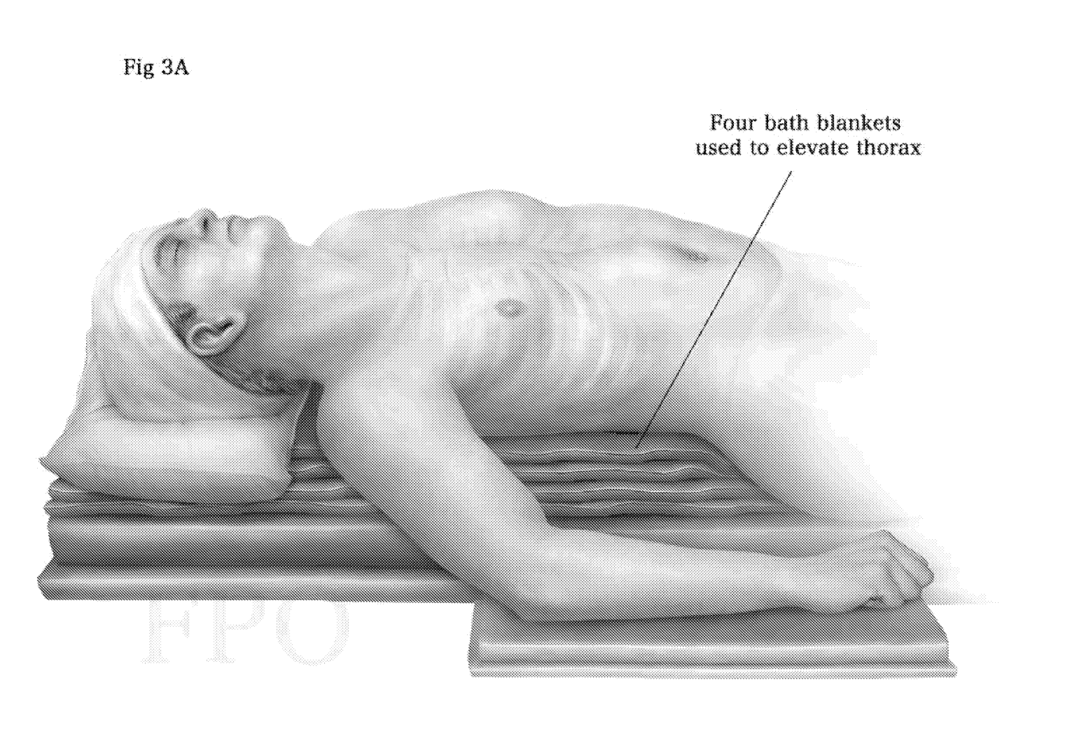

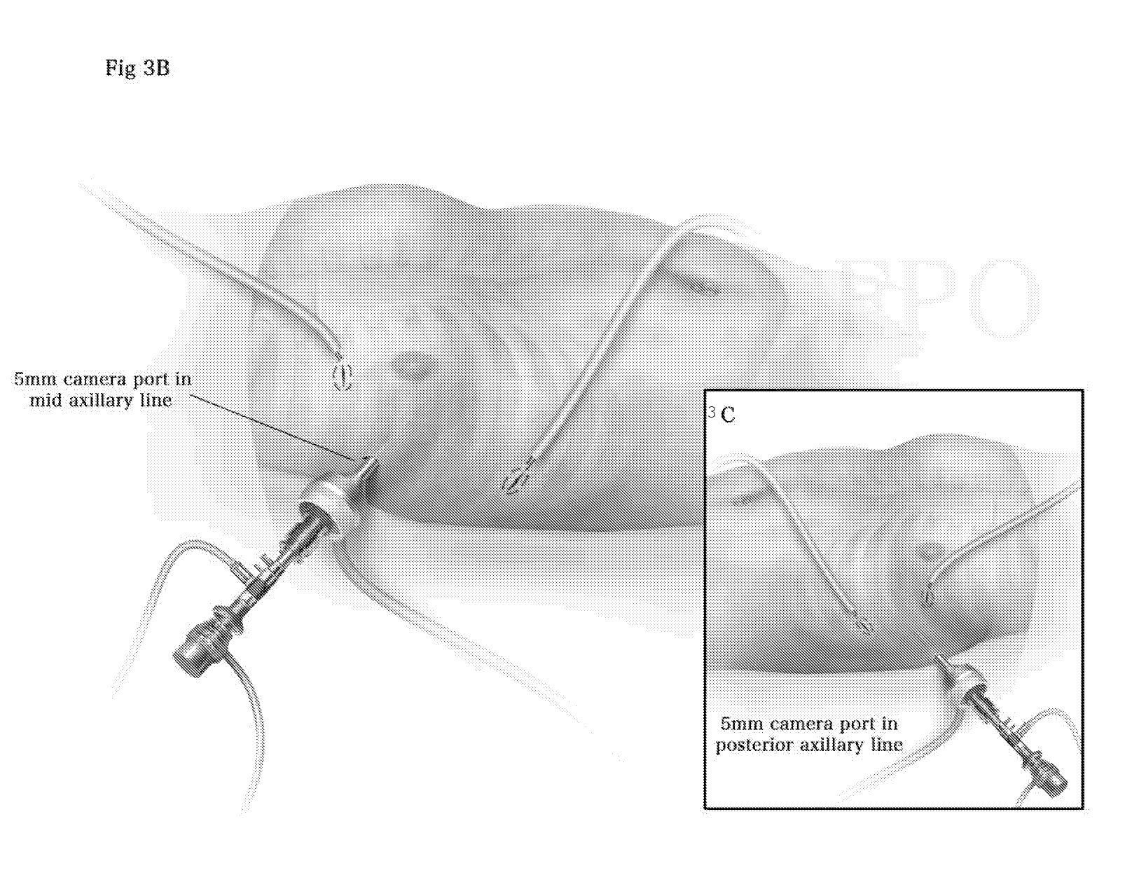

FIG. 3A is an image of the body illustrating the proper positioning of the patient in the operating room, which is critical in order to obtain a successful surgical result. Proper positioning of the patient in the operating room is critical in order to obtain a successful surgical result. The surgeon must have free access to the posterior axillary line bilaterally. Additionally, the use of long thorascopic instrumentation means that the arm must be out of the way so that the instruments can be maneuvered in multiple different planes. Initially, we tried positioning the arm up over the forehead; however, we found that the upper arm was continually in the way, inhibiting movement of the long instruments.

In order to gain access to the posterolateral thorax and have the arms out of the way of instrumentation we elevate the thorax of the patient on three to five bath blankets or other object(s) of similar height. The arms are then placed on slightly padded arm boards on each side of the operating table. The patient is prepped and draped so that the surgeon has access to the entire lateral thorax bilaterally, the sternum, and both groin areas in case they should be needed for urgent access. External defibrillator pads are placed behind the right shoulder and on the left flank. If sterile defibrillator pads are available, one can be placed over the sternum and one directly posterior on the back of the patient.

FIG. 3B and FIG. 3C are images of the heart three ports positioned on the patient illustrating that the surgeon must have free access to the posterior axillary line bilaterally. The present invention provides for performing the operation with three ports. Starting on the right side, a 5 mm port is introduced in the mid-axillary line in the third intercostal space. CO₂ insufflation expands the field and depresses the diaphragm. Using a 30 degree scope, one can obtain good visualization throughout the thorax once absorptive atelectasis of the lung has taken place. The two other ports placed are both 10 mm ports. One is placed in the mid-clavicular line in approximately the second intercostal space and the other in the mid-axillary line in approximately the 7th intercostal space. Both of these ports are placed so that instruments passed through them can access the transverse sinus, behind the superior vena cava and immediately cephalad to the right atrial appendage. This spot can actually be seen “through the pericardium” with a 5 mm scope. To determine the exact placement of the 10 mm port sites, a spinal needle is passed through the chest wall with the tip targeting the transverse sinus. Its position on the chest wall is changed until the site of best access is determined. Once the optimal site for these ports is determined, a 15 mm incision is placed and the 10 mm ports are advanced. Because multiple instrument changes will be made through these port sites, and because insufflation of CO₂ in the chest is critical in order to maintain proper visualization, 0-silk sutures with a Red Rubber Robinson tourniquet are placed around each of these port sites. Then when the ports are later removed to allow introduction of other various sized instruments, the tourniquet can be run down to maintain the insufflation pressure within the thorax. FIG. 3C shows the left side where all ports are placed more posteriorly.

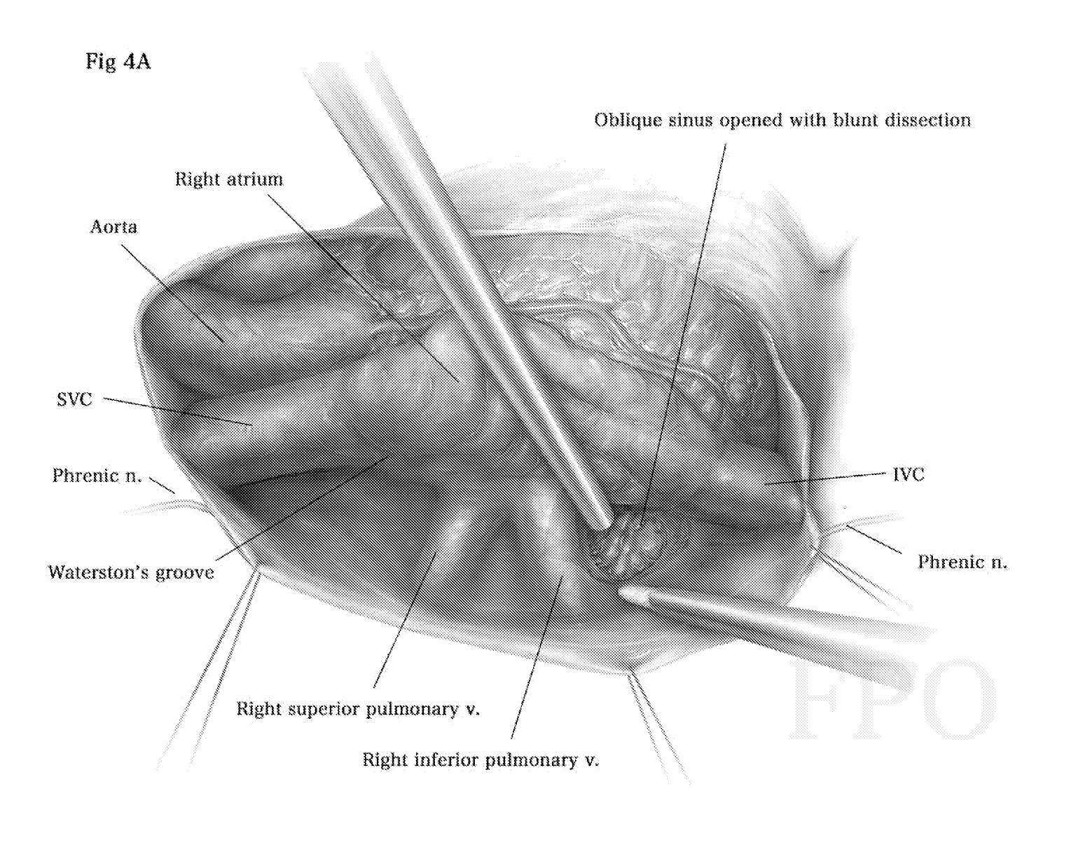

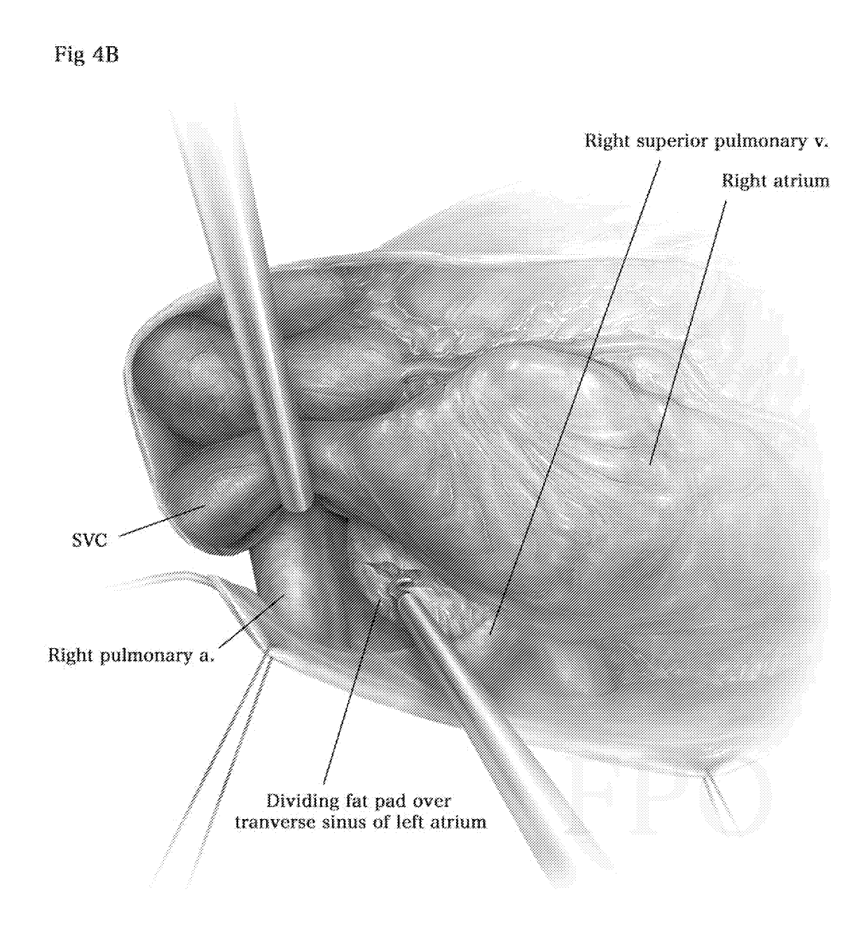

FIG. 4A is an image of the heart illustrating that by working through these port sites, the right pericardium is open from the diaphragm to the level of the aorta approximately 2 cm anterior to the phrenic nerve. Working through these port sites, the right pericardium is open from the diaphragm to the level of the aorta approximately 2 cm anterior to the phrenic nerve. Great care is taken at all times to not stretch the phrenic nerve. Pericardial retraction sutures are placed utilizing an automatic suturing device and brought out through the posterolateral chest wall. Then using two blunt instruments, the fibroareolar tissue between the inferior vena cava and the right inferior pulmonary vein is dissected away until there is a wide opening into the posterior pericardium.

FIG. 4B is an image of the heart illustrating that the pericardium is bluntly and sharply dissected away until a portion of the superior vena cava is converted to intrapericardial. Attention is then directed to the space between the right superior pulmonary vein, the right pulmonary artery, and the superior vena cava. The pericardium is bluntly and sharply dissected away until a portion of the superior vena cava is converted to intrapericardial. Then using a suction irrigator for retraction in the left hand and an L cautery device in the right hand, the tissue between the superior vena cava and the right pulmonary artery are divided along the entire course of the superior vena cava. Having thus surrounded the superior vena cava, one should be able to elevate it and see through the transverse sinus with a 5 mm scope.

The visceral pericardium surrounds the superior vena cava (SVC) and behind it inserts onto the muscular dome of the left atrium. Behind the SVC, between the two leafs of investing pericardium as they insert on the dome of the left atrium, there is always a very well-established fat pad behind the superior vena cava and in front of the left atrial muscular dome. This fat pad must be dissected away so that the ablation devices can get down to the muscular dome of the atrium. Fat is a relative insulator to radiofrequency energy. If one tries to burn through this fat, this is a common site of a gap in the ablation line leading to a failure of the procedure. Accordingly, it is critical that this fat pad be dissected until the operator can see the muscular dome of the left atrium.

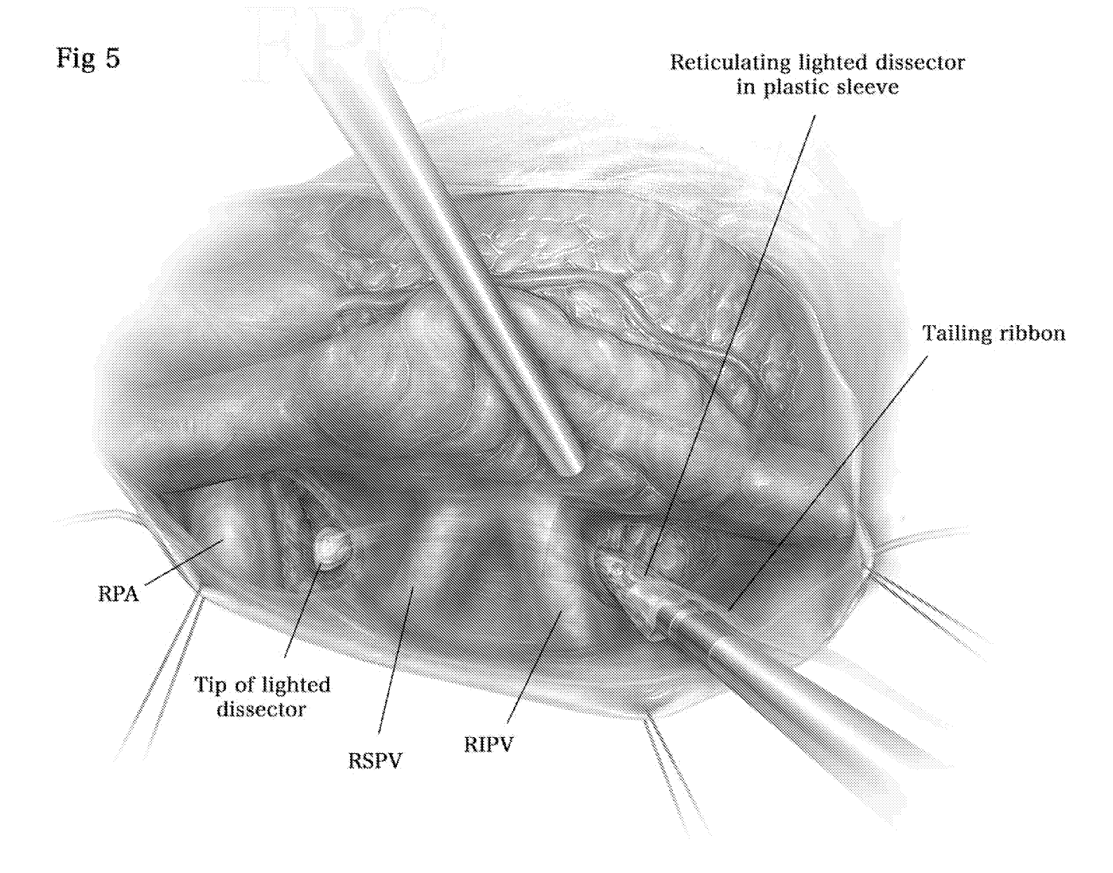

FIG. 5 is an image of the heart illustrating that the lighted dissector will not fit through the 10 mm port, but will easily slide through the port site. At this point in time, the 10 mm port that is in the most caudad site is removed. The lighted dissector (AtriCure, Cincinnati, Ohio) will not fit through the 10 mm port, but will easily slide through the port site. Once it is introduced into the chest, the skin tourniquet is run down in order to reestablish the CO₂ pressure within the chest and ensure ongoing exposure and visualization. This lighted dissector is advanced through the space between the right inferior pulmonary vein and the inferior vena cava into the posterior pericardium. Then by slowly and carefully articulating the clamp, the lighted blunt tip is dissected through the space between the right pulmonary artery and the right superior pulmonary vein. The clear plastic sheath is pulled off of the dissector and the lighted dissector is withdrawn.

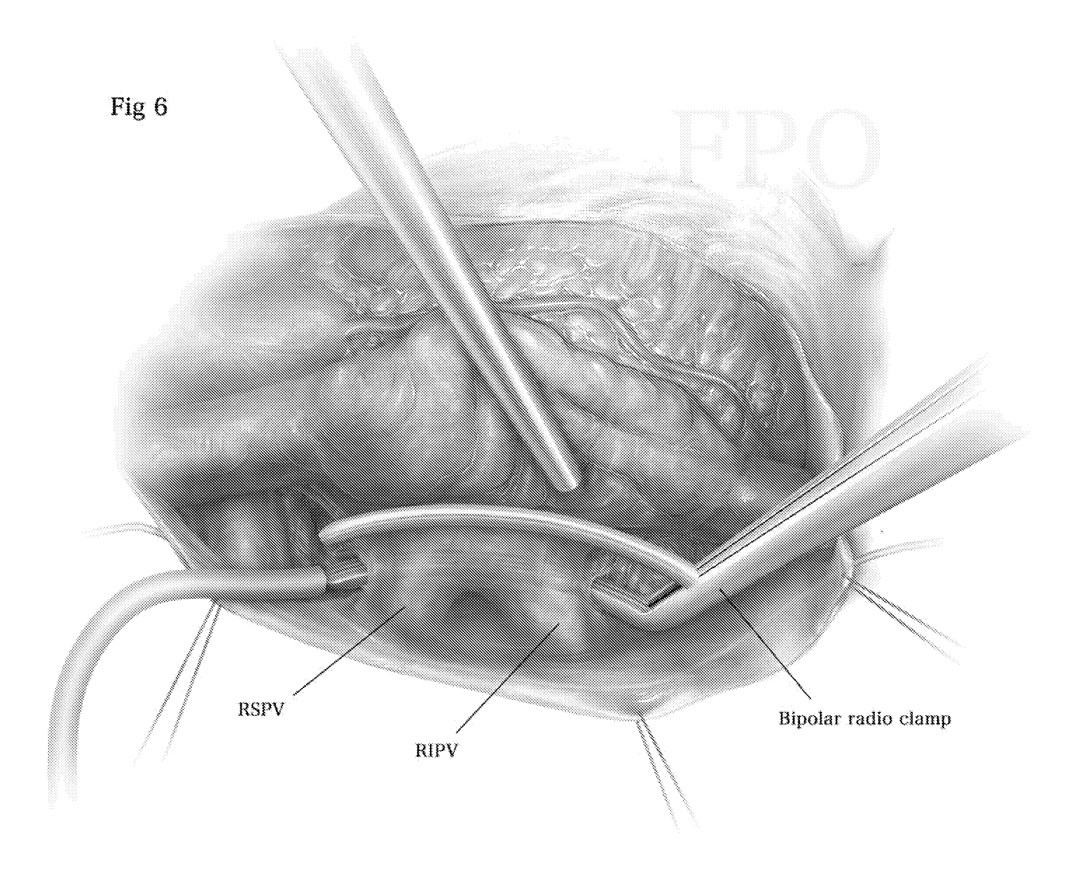

FIG. 6 is an image of the heart illustrating that a sensing pen is passed through the most cephalad port site and used to obtain a baseline electrogram on multiple sites on both the superior and inferior pulmonary veins. This clear plastic tape is merely a leader, the other end of which attaches to the posterior jaw of a bipolar radiofrequency ablation clamp (AtriCure, Cincinnati, Ohio). At this point, a sensing pen is passed through the most cephalad port site and used to obtain a baseline electrogram on multiple sites on both the superior and inferior pulmonary veins. These baseline electrograms should show transmitted electrical activity from the atrium. After the ablation lines have been placed, the electrograms in the pulmonary veins are again obtained and this time, the absence of transmitted electrical activity from the atrium indicates acute entrance block has been obtained. Alternatively, if the patient is in sinus rhythm, one can pace in the pulmonary veins and look for exit block. The pen is also utilized to deliver high frequency stimulation to locate the presence of active ganglionated plexi. These are located by uncovering the bradycardic response, e.g., greater than 50% increase in R-to-R interval in response that occurs when one stimulates right over a ganglionated plexi [11]. The closed bipolar clamp is then introduced through the most-caudad port site and then the tourniquet is again snuggled around the shaft of the camp to reestablish the pneumothorax. By pulling the clear plastic tape up and out through the second intercostal space port site, the posterior jaw of the clamp is then delivered behind the right-sided pulmonary veins. It is manipulated until the jaws are well-up on the antrum of the pulmonary vein and well-away from the bifurcation of the pulmonary veins. Three to five firings of the clamp are then done to produce an ablation line on the right pulmonary vein antrum. After the ablation lines are completed with the clamp, the pen is again utilized to test in areas that were previously positive. If they are still positive, indicating they were not ablated with the clamp, this same sensing pen is utilized to deliver radiofrequency energy performing further ablation until all the areas that were positive for ganglionated plexi are now negative.

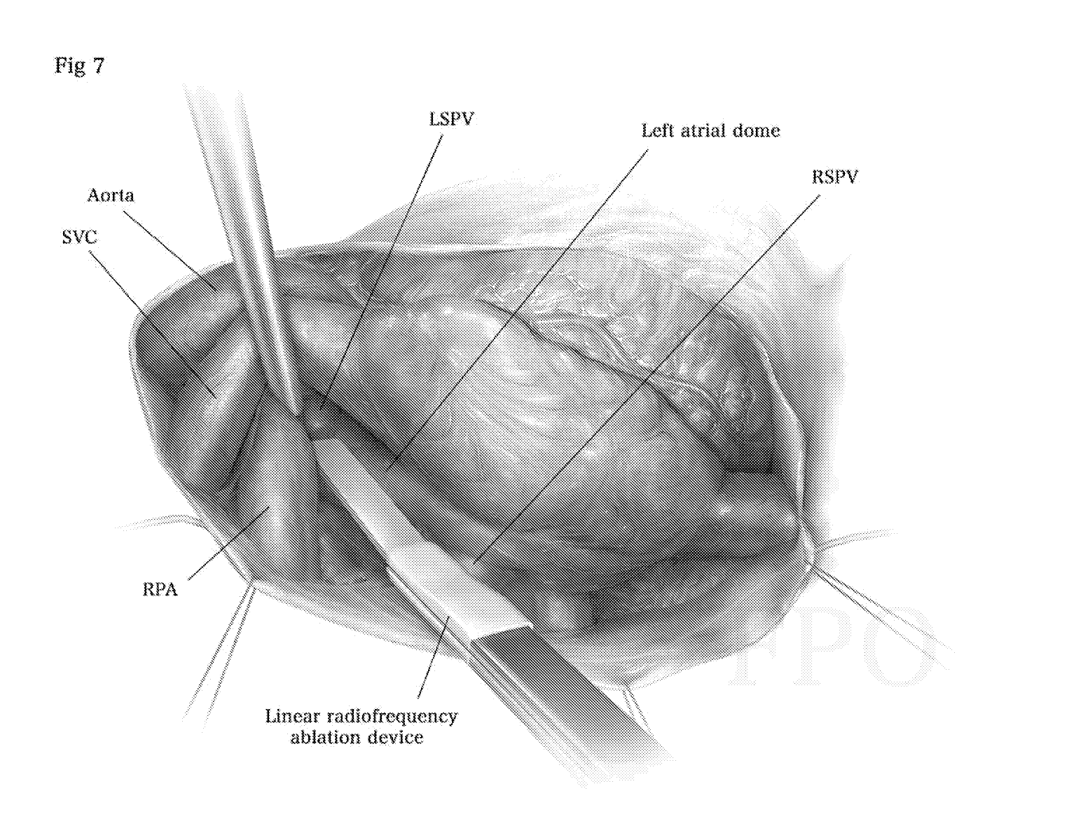

FIG. 7 is an image of the heart illustrating the internally cooled, linear bipolar radiofrequency device introduced through the most caudad port site. The internally cooled, linear bipolar radiofrequency device is now introduced through the most caudad port site. This device (e.g., Cooled Rails, AtriCure, Cincinnati, Ohio) has a deflectable tip and a malleable shaft. Now that the fat pad behind the superior vena cava has been divided, one can clearly see the muscular dome of the left atrium when the superior vena cava is elevated. The linear bipolar ablation device is positioned behind the superior vena cava. It is utilized to make a linear burn from the right superior pulmonary vein across the dome of the left atrium pointing towards the left superior pulmonary vein. In most cases when looking through the transverse sinus, the left superior pulmonary vein and even left atrial appendage can easily be visualized through this approach. This ablation line from the right superior pulmonary vein towards the left superior pulmonary vein is placed as posteriorly on the dome of the atrium as is possible. Some times fibroareolar connective tissue between the atrial dome and the right pulmonary artery must be divided in order to allow this posterior placement of this ablation line.

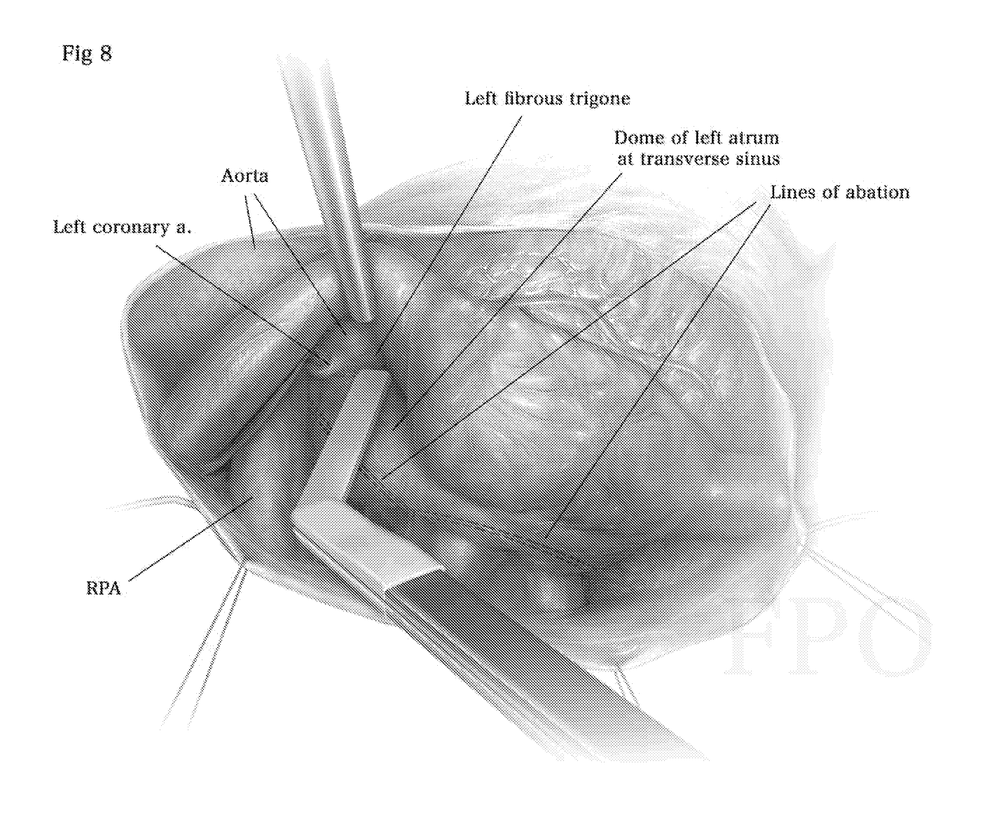

FIG. 8 is an image of the heart illustrating the tip of the ablation device articulated to the right and further bending of the malleable shaft with the tip positioned at the junction of the noncoronary cusp and the left coronary cusp of the aorta. The tip of this same ablation device is now articulated to the right and further bending of the malleable shaft done if possible. The tip is positioned at the junction of the noncoronary cusp and the left coronary cusp of the aorta. This juncture as well as the left main coronary can usually be easily visualized by placing the 30 degree 5 mm scope behind the superior vena cava and into the transverse sinus. The operator will see that the left main coronary artery is 2-to-3 cm away from the site of this ablation. Confirmation of the location of the tip of the ablation device can be done by looking at the transesophageal echo. Utilizing the mid-esophageal, long access, 140 degree view, the left fibrous trigone where the aortic annulus touches the mitral annulus can easily be visualized. Slight wiggling of the ablation device produces movement which is readily apparent on the echo view when the device is in the proper position. A lesion is then burned from the fibrous trigone obliquely on the dome so that it connects the left fibrous trigone to the transverse ablation line across the dome of the atrium. This linear ablation line can be touched-up by using the bipolar pen to also deliver radiofrequency ablation lesions in a “stamping” fashion. This completes the lesions that are placed from the right thorascopic approach. The pericardium is then closed, a long catheter (I-Flow Corporation) to continuously infuse Marcaine is tunneled beneath the pleura to bathe all the intercostal nerves, a 19 French silastic chest tube is placed, the ports are withdrawn, the lung inflated, and all the wounds closed.

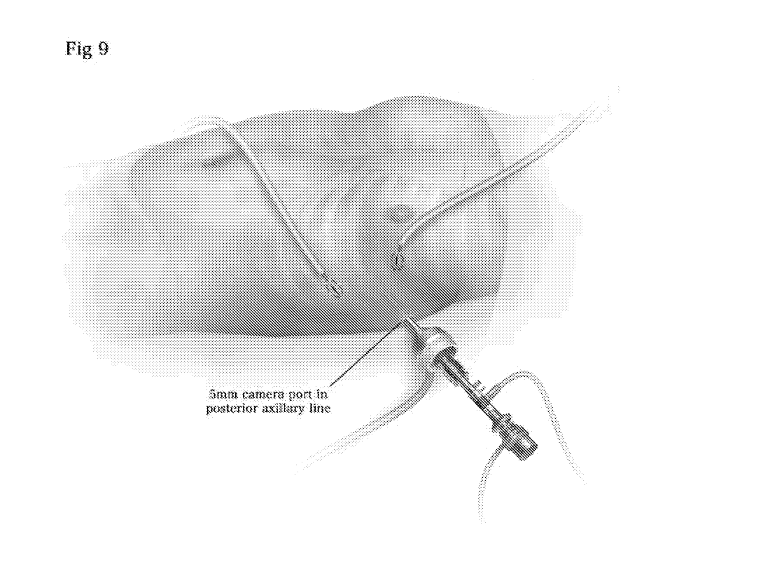

FIG. 9 is an image of the heart three ports positioned on the left side of the patient that is similar to the right side, but somewhat more posterior. The approach is similar to the right side, but somewhat more posterior. The 5 mm camera port is placed in the third intercostal space between the mid axillary line and the posterior axillary line and CO₂ is insufflated. Again, spinal needles are advanced through the chest to determine the exact positioning of the other two ports so that their site will afford access to the left atrial appendage and the transverse sinus. Usually, the cephalad port is in the midclavicular line at approximately the second intercostal space and the caudad port is in the sixth or seventh intercostal space at the posterior axillary line.

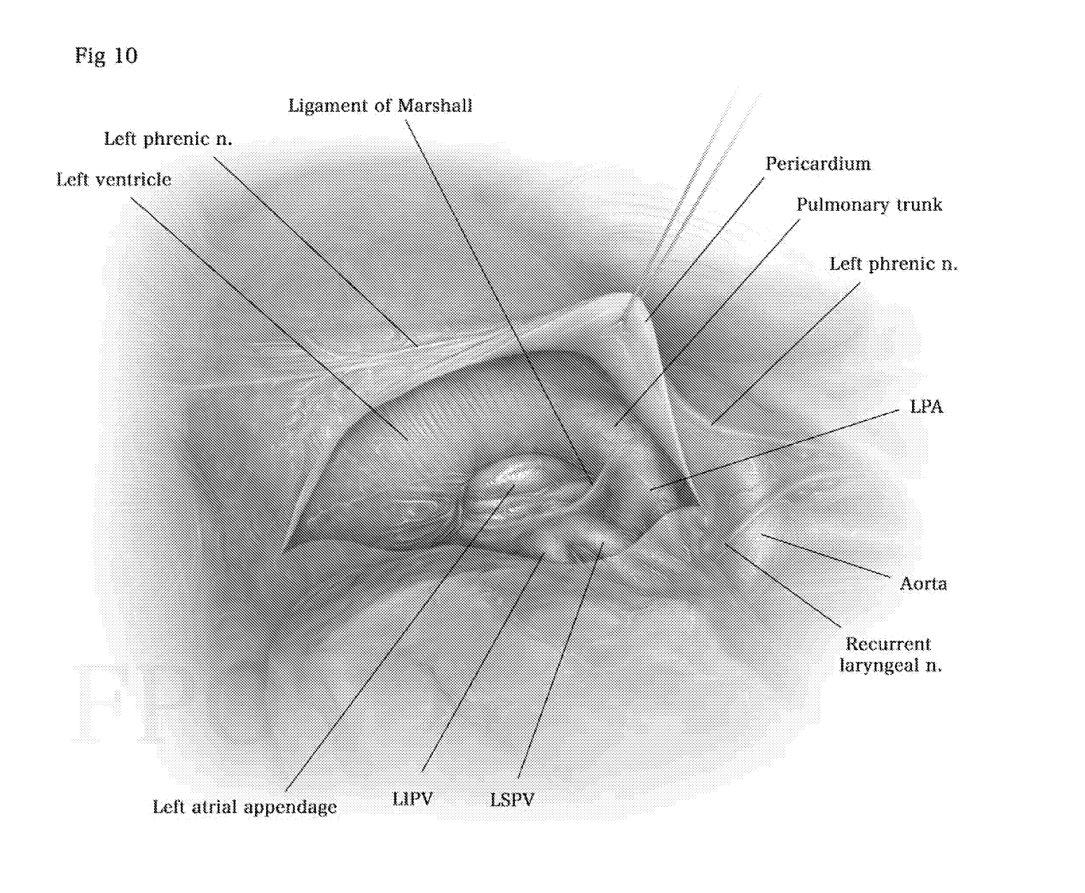

FIG. 10 is an image of the heart illustrating that the pericardium is posterior to the phrenic nerve. Working through these ports, the pericardium is opened. However, on the left side, the opening to the pericardium is done posterior to the phrenic nerve. Great care must be taken to avoid injury to the phrenic nerve. When extending the incision cephalad care must also be taken not to, injure the recurrent laryngeal nerve as it courses beneath the aorta. To protect the phrenic nerve, a single suture is placed in the anterior leaf of the pericardium, just posterior to the phrenic nerve, and this is brought out through the anterior chest wall. This lifts the phrenic nerve out of the field and also helps to expose the atrial appendage.

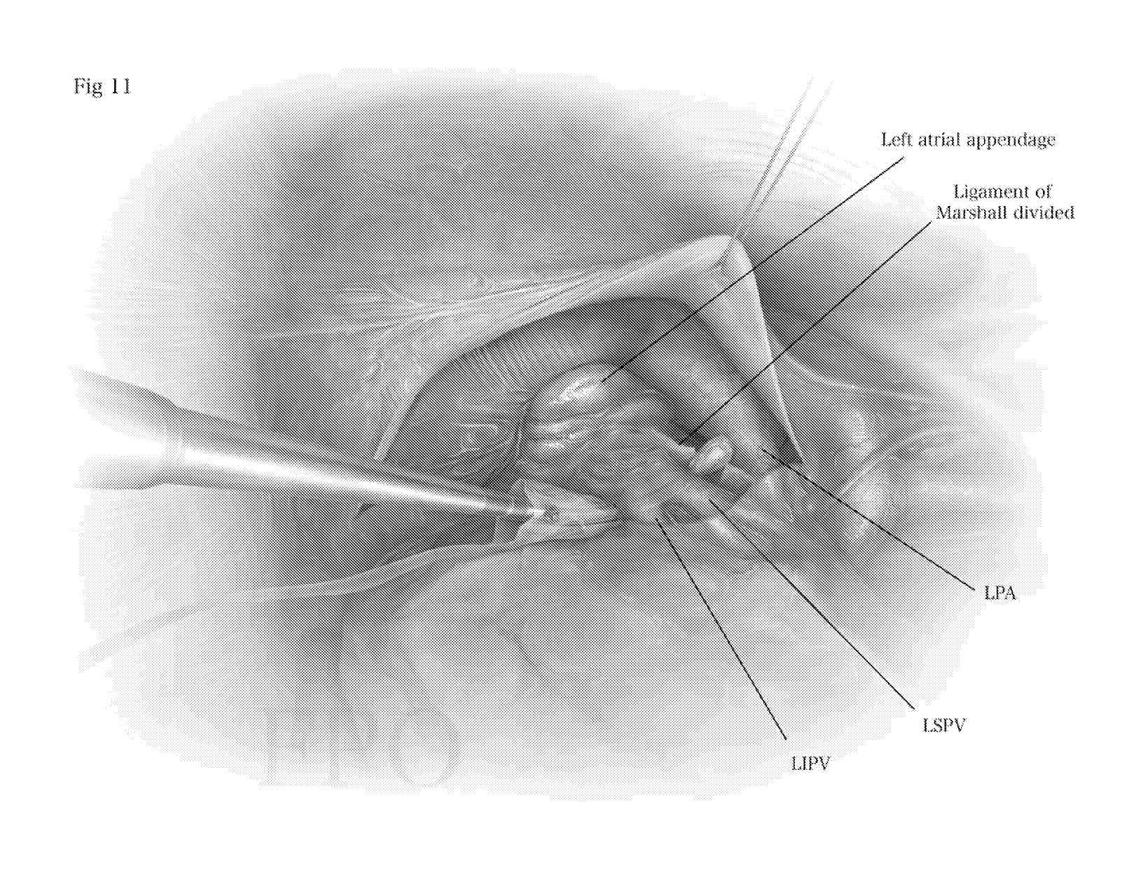

FIG. 11 is an image of the heart illustrating the transverse sinus and the lesions placed from the right side. Working through these ports, one should be able to visualize into the transverse sinus and see the lesions that were placed from the right side. The sensing pen is placed and baseline electrograms in the pulmonary veins are recorded. Then high frequency stimulation is performed to locate any active ganglionated plexi and their positions are noted. Following this, the ligament of Marshall is divided all the way posterior. Thereafter, the lighted dissector is introduced from the most caudad port site and directed around the pulmonary veins with the tip coming up at the point of the divided ligament of Marshall.

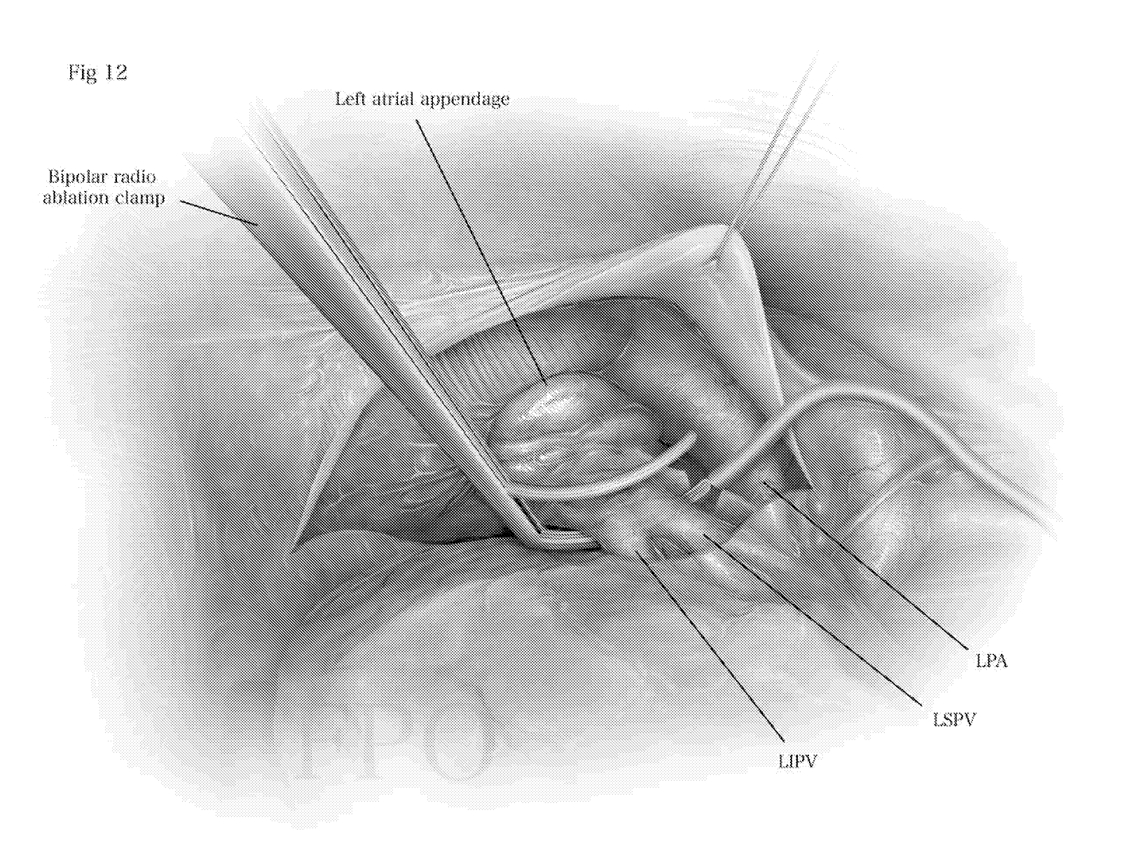

FIG. 12 is an image of the heart illustrating the bipolar radiofrequency clamp introduced through the most caudad port site. The bipolar radiofrequency clamp is then introduced through the most caudad port site. Using the guide that was attached to the lighted dissector, the posterior jaw is introduced behind the pulmonary vein and the clamp is closed well up on the pulmonary vein antrum where three separate firings are performed changing the position of the clamp each time. The clamp is withdrawn and the pen is again introduced. Sensing in the pulmonary veins should now show electrical silence indicating entrance block so that no atrial electrical activity is transmitted into the veins. Alternatively, if the patient is in sinus rhythm, one can pace in the pulmonary veins and look for exit block. Finally, the pen is used to stimulate in any areas where active ganglionated plexi were previously located. If these sites have not been ablated by the application of the radiofrequency clamp, they are now ablated by applying radiofrequency energy through the pen device.

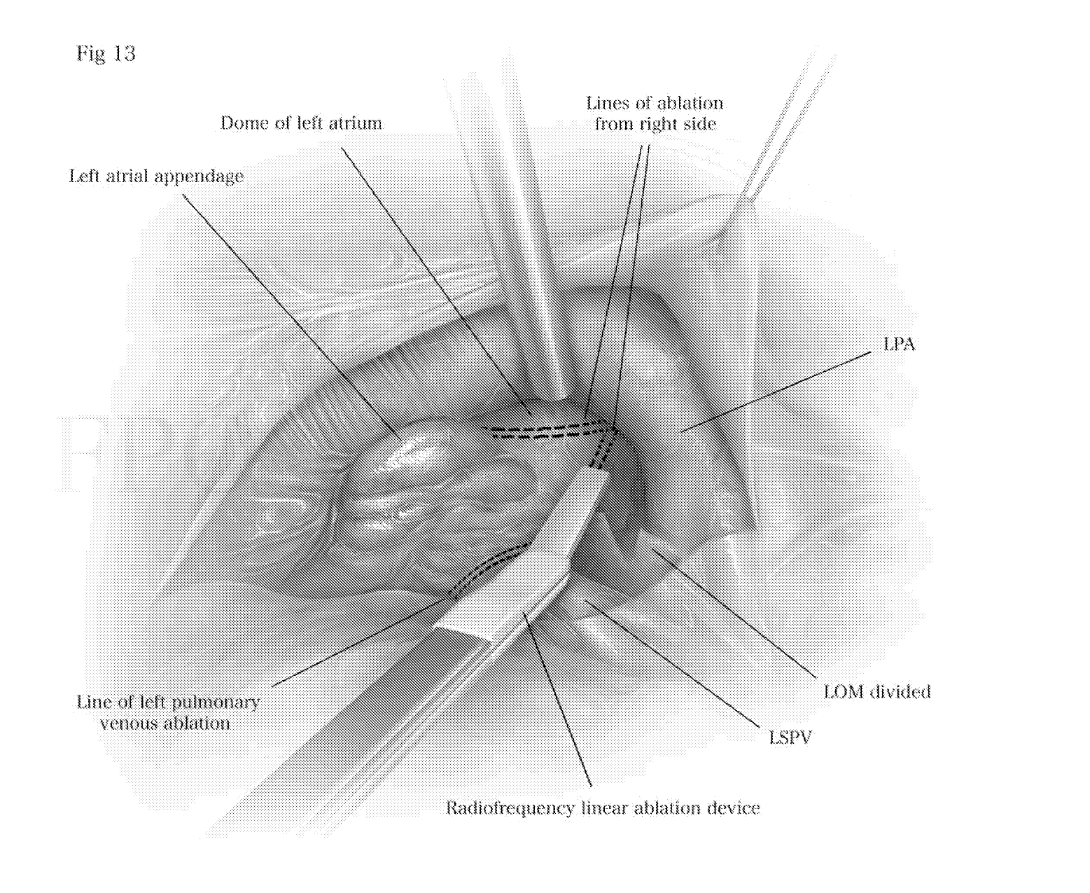

FIG. 13 is an image of the heart illustrating that by The clamp is withdrawn and the linear radiofrequency ablation device is introduced through the most caudad port site. The clamp is withdrawn and the linear radiofrequency ablation device is introduced through the most caudad port site. The atrial appendage and the pulmonary artery are retracted to open up visualization into the transverse sinus. One can readily see the dome line coming across from the right side as it is aimed towards the left superior pulmonary vein. The linear ablation device is now used to complete this dome line so that it connects the right superior pulmonary vein over to the left superior pulmonary vein as far posteriorly as can be done in a transverse sinus. The malleable linear ablation device is now articulated to the left and bent to the left. Another ablation line is now placed from the left fibrous trigone to the right superior pulmonary vein.

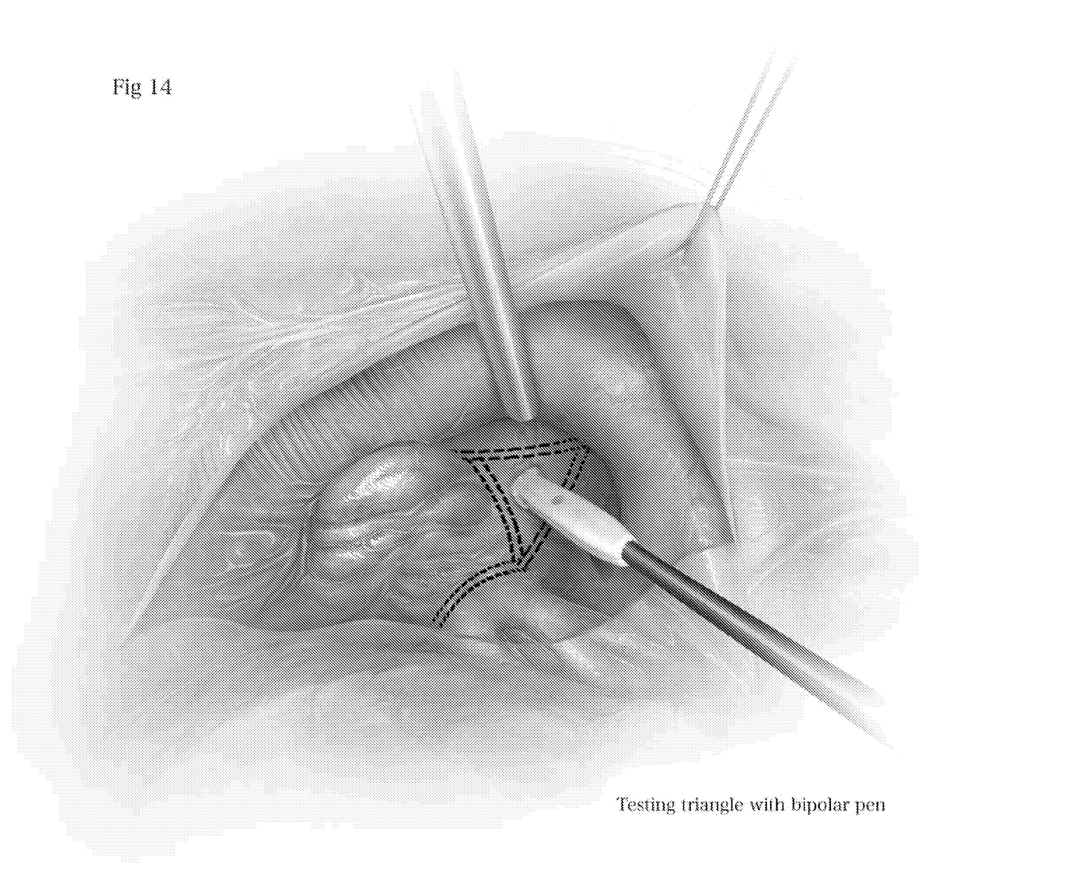

FIG. 14 is an image of the heart illustrating the ablations that have been done on the dome and an inverted triangle constructed. With the ablations that have been done on the dome, an inverted triangle has now been constructed. The sensing pen is placed into the triangle. If each of these ablation lines is transmural, then there will be no conducted atrial activity into this triangle and the recorded electrogram will be flat. Alternatively, if the patient is in sinus rhythm, one can pace in this triangle and look for exit block to confirm that acute block has been obtained.

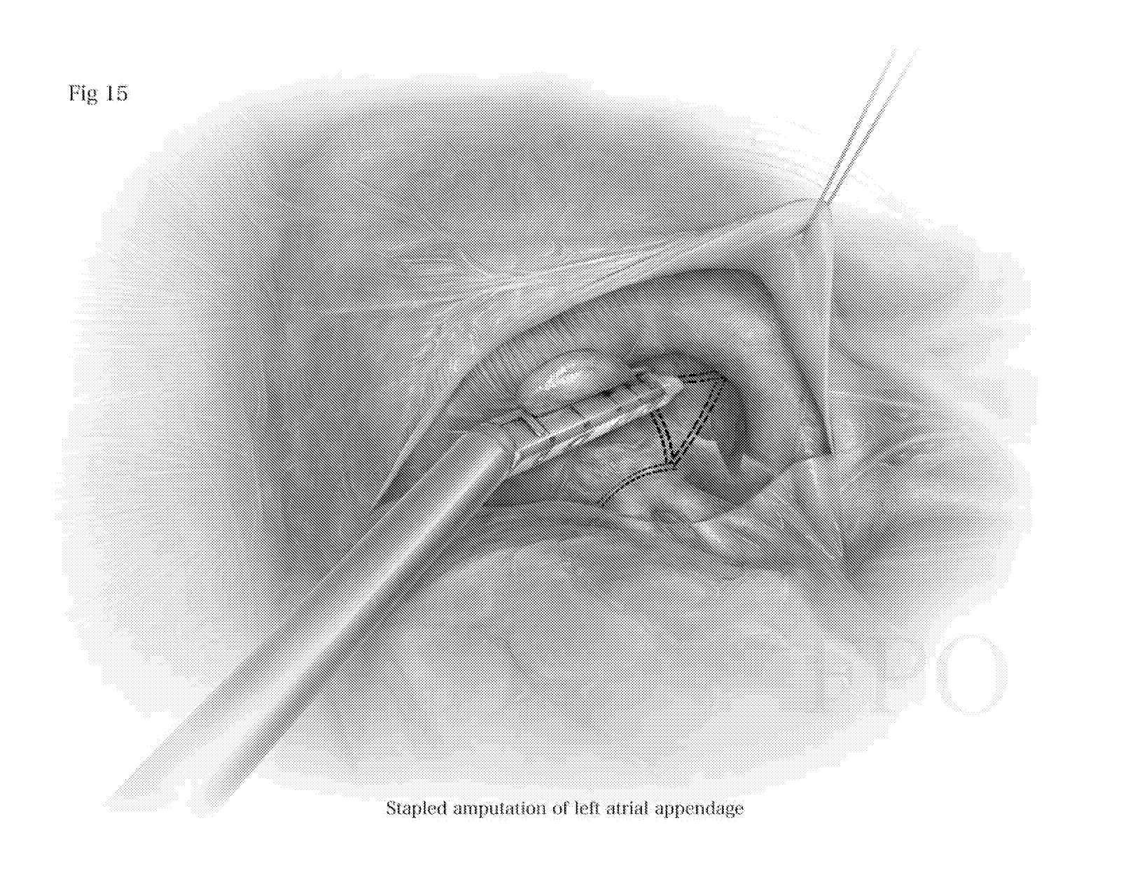

FIG. 15 is an image of the heart illustrating a stapling device introduced through the most caudad port site and positioned around the base of the atrial appendage. Finally, a stapling device is introduced through the most caudad port site and carefully positioned around the base of the atrial appendage. Using the transesophageal echo to help guide the placement, the stapler is closed on the base of the atrial appendage and it is amputated. The amputated atrial appendage is withdrawn with the stapler. An infusion catheter is then threaded subpleural from caudad to cephalad to allow continuous infusion of Marcaine along all the intercostal nerve roots. The pericardium is reapproximated with a single stitch, a chest tube is placed, and all of the wounds are closed in layers. Sterile dressings are applied, the patient is awakened, extubated in the operating room, and transferred to the post operative care ward.

The multiple burns that have been done usually result in producing an effusive pericarditis. To limit this, and allow early removal of the pleural catheters, all patients are started on steroids in the operating room and continued on an oral form for three to five days post-operatively. All pre-operative medications, including antiarrhythmics are reinstated. Unless contraindicated, Coumadin is started on all patients and the dosing is refined as an outpatient. Most patients are discharged on the third postoperative day. Patients are seen at one, three, and six months post-operatively. The one month visit is for would checks. At the 3 month visit a 24 hour Holter is done. If the Holter shows no atrial fibrillation, atrial tachycardia and no atrial flutter, and the patients reports no episodes of the same, the antiarrhythmic medications are discontinued. At the six month visit, a 2 week monitor is done. If the 2 week monitor shows no atrial fibrillation, atrial tachycardia and no atrial flutter, the coumadin is discontinued unless it is specifically indicated for another reason. A 2-week monitor is done at one year postoperatively and at 12 month intervals thereafter for life. If the patient has any recurrence of atrial dysrhythmia, he is referred for full electrophysiological study in the EP lab.

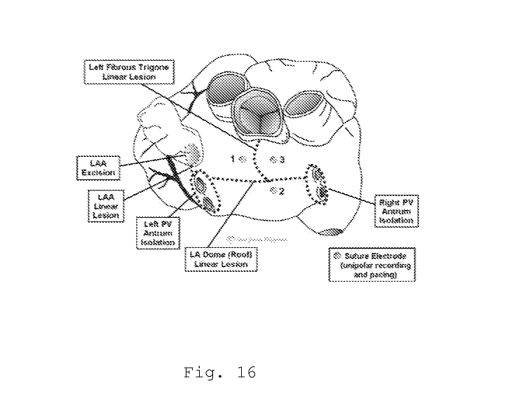

In the minimally invasive approach, excellent visualization through the transverse sinus behind the aorta and the pulmonary artery is obtained. Therefore, the present invention provides placing all of our connecting lesions here on the dome of the left atrium. FIG. 16 is a schematic of the location of the connecting lesions on the dome of the atrium and position of the temporary pacing/recording electrodes. By working behind the superior vena cava and through the transverse sinus, a transverse connecting lesion across the dome of the left atrium connecting the right superior PV with the left superior PV can be formed. It is then only a short extension of this line on the left side that connects it to the base of the left atrial appendage. The connecting lesion to the mitral valve annulus can also be accomplished on the dome of the left atrium within the transverse sinus. The left fibrous trigone connects the mitral valve annulus to the aortic valve annulus at the aortic root. The left fibrous trigone meets the aortic valve at a point where the left coronary cusp and the non-coronary cusp join. Therefore, with good visualization, we can place this connecting lesion from the left fibrous trigone at the anterior mitral valve annulus, across the anterior dome of the atrium to the transverse dome line. This line connecting to the fibrous triangle is made medially towards the center of the atrial dome. If it is made laterally on the right side of the dome, it may cross the sinus node artery which can affect activation of the left atrium during sinus rhythm, resulting in temporary sinus node dysfunction. The bilateral PV antrum isolation and connecting linear lesions then completes the left atrial lesions equivalent to the classic Cox Maze III operation. Although this lesion set can be accomplished with any energy source that creates transmural lesion, we have used radiofrequency. The pulmonary vein antra are isolated using 3 applications of a bipolar RF clamp (Isolator Synergy Ablation Clamp, Atricure, Inc, Cincinnati, Ohio) at an average power of 30 watts. The linear lesions are made with a combination of a short (10 sec application Atricure Isolator Multifunctional Pen) and a long (40 sec application) of the Coolrail Linear Pen, Atricure Inc, (Cincinnati, Ohio).

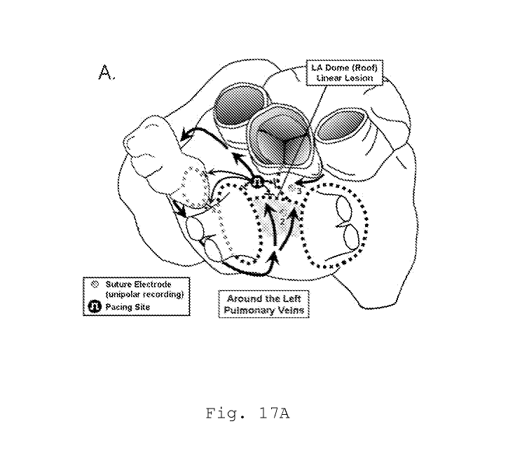

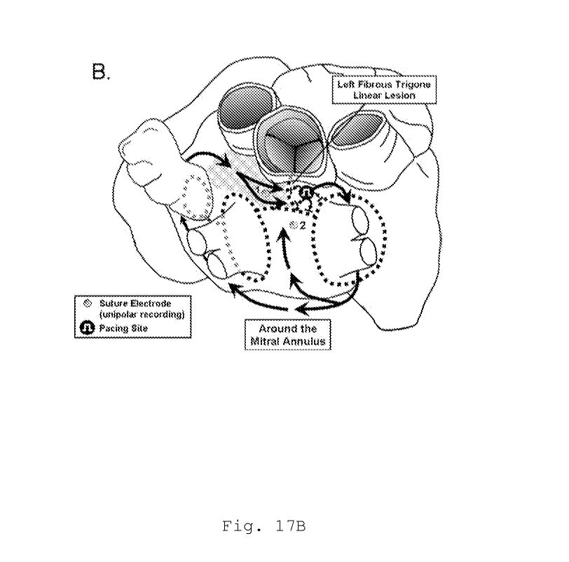

It is essential to demonstrate activation block across all surgically created lesions. created lesions. Unlike the cut and sew technique, it often requires several epicardial radiofrequency applications to achieve activation block, e.g., the radiofrequency energy three times and then test for block. After isolating the PV antra, recordings are obtained along the PVs and the atrium between the isolating lesions and the PVs to demonstrate absence of atrial potentials (entrance block). If isolation has not been achieved, 1-2 more applications of the bipolar RF clamp are made, and then it is retest for block. Demonstrating block along linear lesions is more difficult and requires determining the activation time at several sites on one side of the lesion line while pacing on the other side (activation sequence). In order to do this, 3 temporary wire electrodes are placed on the left lateral side of the trigone line at the base of the left atrial appendage (electrode 1 in FIGS. 16 and 17), one on the posterior side of the transverse dome line (electrode 2), and one on the medial side of the trigone line (electrode 3).

FIG. 17A is a schematic indicating the electrode placement to verify activation block across the transverse dome line. Atrial pacing is initiated anterior to the ablation line at the base of the left atrial appendage (electrode 1) with the activation time to electrode 2 recorded as reference. If activation block is complete, the activation must propagate around the left pulmonary veins (arrows). As a result, activation times measured at several locations in the stippled area will increase as the probe is moved towards the linear lesion. FIG. 17B is a schematic indicating the electrode placement to confirm complete block across the left fibrous trigone line. Pacing is performed on the right side of the trigone line (electrode 3) and activation sequence is determined by recording the activation times to electrode 1 and several sites in the stippled area (using the probe electrode). Activation block is confirmed by measuring increased activation times as the probe is moved medially towards the trigone line along its length.

FIG. 17A is a schematic of the electrode placement to verify activation block across the transverse dome line. Atrial pacing is initiated anterior to the ablation line at the base of the left atrial appendage (electrode 1) with the activation time to electrode 2 recorded as reference. If activation block is complete, the activation must propagate around the left pulmonary veins (arrows). As a result, activation times measured at several locations in the stippled area will increase as the probe is moved towards the linear lesion. Activation block across the transverse dome line can be verified by atrial pacing at the base of the left atrial appendage anterior to the ablation (electrode 1) and measuring the activation time to fixed electrode 2. Then, using a probe electrode to map the posterior left atrium (indicated as the gray stippled area in FIG. 17A) activation times are recorded to show that activation is moving superiorly, towards the dome line (arrows in stippled area). With complete block, activation times will increase as the probe gets closer to the ablation line. Any area towards the lesion showing a decrease in activation time would indicate incomplete block (gap in the lesion). Confirmation of block across the trigone line can be verified by pacing on the right side of the line (electrode 3) and mapping the region on the left side of the trigone line (indicated as the gray stippled area in FIG. 17B) using the probe electrode, comparing activation times to that recorded to the fixed electrode (electrode 1). Activation should be propagating superiorly and medially towards the trigone line along its length (indicated as the arrows in stippled area in FIG. 17B), indicating no break in the line.

We have now performed this lesion set using minimal access techniques, in over 50 patients with persistent and longstanding persistent AF. Initially we utilized bilateral 6 cm intercostal working incisions (without rib spreading). In the last ten patients, we performed this lesion set with a totally thorascopic techniques using a 5 mm port and two 10 mm ports bilaterally. There has been no mortality. In all of these patients we were able to complete the lesion set and demonstrate complete block across the lesion lines.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Wolf R K, Schneeberger E W, Osterday R, Miller D, Merrill W, Flege J B, Gillinov A M. Video assisted bilateral pulmonary vein isolation and left atrial appendage exclusion for atrial fibrillation. J Thorac Cardiovasc Surg 2005; 130(3):797-802.
2. Puskas J, Lin E, Bailey D, Guyton R. Thorascopic radiofrequency pulmonary vein isolation and left atrial appendage exclusion. Ann Thorac Surg 2007; 83: 1870-72.
3. Yilmaz A, Van Putte B P, Van Boven W J. Completely thorascopic bilateral pulmonary vein isolation and left atrial appendage exclusion for atrial fibrillation. J Thorac Cardiovasc Surg 2008; 136: 521-22.
4. Gillinov A M, Bhavani S, Blackstone E H, Rajeswaran J, Svensson L G, Navia J L, Pettersson B G, Sabik J F III, Smedira N G, Mihaljevic T, McCarthy P M, Shewchik J, Natale A. Surgery for permanent atrial fibrillation: impact of patient factors and lesion set. Ann Thorac Surg 2006; 82(2):502-13; discussion 513-4.
5. Wijffels M C, Kirchhof C J, Dorland R, Power J. Allessie M A. Electrical remodeling due to atrial fibrillation in chronically instrumented conscious goats: roles of neurohumoral changes, ischemia, atrial stretch, and high rate of electrical activation. Circulation 1997; 96(10):3710-20.
6. Hu H, Sachs F. Stretch-activated ion channels in the heart. J Mol Cell Cardiol 1997; 29(6):1511-23.
7. Sakai R, Hagiwara N, Kasanuki H, Hosoda S. Chloride conductance in human atrial cells. J Mol Cell Cardiol 1995; 27(10):2403-8.
8. Shinagawa K, Shi Y F, Tardif J C, Leung T K, Nattel S. Dynamic nature of atrial fibrillation substrate during development and reversal of heart failure in dogs. Circulation 2002; 105(22):2672-8.
9. Nattel S, Shiroshita-Takeshita A, Cardin S, Pelletier P. Mechanisms of atrial remodeling and clinical relevance. Curr Opin Cardiol 2005; 20(1):21-5.
10. Shinbane J S, Lesh M D, Stevenson W G, Klitzner T S, Natterson P D, Wiener I, Ursell P C, Saxon L A. Anatomic and electrophysiologic relation between the coronary sinus and mitral annulus: implication for ablation of left-side accessory pathways. Am Heart J 1998; 136:93-98.
11. Vincenzi F F, West T C. Release of autonomic mediators in cardiac tissue by direct subthreshold electrical stimulation. J Pharmacology and Experimental Therapeutics 1963; 141:185-94.

What is claimed is:

1. A method of forming atrial lesions on the dome of the atrium comprising the steps of:
positioning the patient on a table;
placing an object under a thorax area of the patient to raise the thorax 3 to 18 inches to gain access to a posterolateral thorax area;
preparing access to a lateral thorax area bilaterally and a sternum area of the patient;
placing one or more external defibrillator pads on the patient;
inserting a first right port in a mid-axillary line in the third intercostal space of the patient;
insufflating the patient with CO₂ to expand the field and depresses the diaphragm;
inserting a second right port in a mid-clavicular line in approximately a second intercostal space of the patient;
inserting a third right port in a mid-axillary line in approximately the 7th intercostal space of the patient, wherein the second right port and third right port are positioned most cephalad and the other is positioned most caudad so that an instrument can access the transverse sinus, behind the superior vena cava and immediately cephalad to the right atrial appendage;
sealing at least partially the first right port, the right second port and the third right port;
dissecting the fibroareolar tissue between the inferior vena cava and the right inferior pulmonary vein to form an opening into the posterior pericardium;
converting a portion of the superior vena cava to an intrapericardial area;
dividing the tissue between the superior vena cava and the right pulmonary artery;
dissecting the fat pad located behind the superior vena cava and in front of the left atrial muscular dome to expose the atrium muscular dome;
removing the second right port or the third right port and insert a lighted dissector;
advancing the lighted dissector between the right inferior pulmonary vein and the inferior vena cava into the posterior pericardium;
dissecting through the space between the right pulmonary artery and the right superior pulmonary vein with the lighted dissector;
removing the lighted dissector;
inserting a sensing pen through the most cephalad port;
obtaining a right baseline electrogram on multiple sites on both the superior and inferior pulmonary veins with the sensing pen;
introducing a closed bipolar clamp comprising a jaw and a posterior jaw a through the most caudad port site;
positioning the posterior jaw of the clamp behind the right-sided pulmonary veins until the jaw and the posterior jaw are well-up on the antrum of the pulmonary vein and well-away from the bifurcation of the pulmonary veins;
producing an ablation line on the right pulmonary vein antrum by firing the clamp 2-6 times;
obtaining a second right electrogram on multiple sites on both the superior and inferior pulmonary veins with the sensing pen, wherein the absence of transmitted electrical activity from the atrium indicates acute entrance block has been obtained;
introducing a linear bipolar radiofrequency device through the most caudad port;
positioning the linear bipolar ablation device behind the superior vena cava;
forming a first linear ablation line from the right superior pulmonary vein across the dome of the left atrium pointing towards the left superior pulmonary vein;
positioning the linear bipolar ablation device at the junction of the noncoronary cusp and the left coronary cusp of the aorta;
forming a second linear ablation line from the fibrous trigone obliquely on the dome to connect the left fibrous trigone to the transverse ablation line across the dome of the atrium;
closing the pericardium;
withdrawing the first right port, the second right port and the third right port;
closing a first right port incision, a second right port incision and a third right port incision;
inflating the lung;
positioning a first left port in a mid-axillary line in the third intercostal space of the patient;
insufflating the patient with CO₂ to expand the field and depresses the diaphragm;
positioning a second left port in a mid-clavicular line in approximately a second intercostal space of the patient;
positioning a third left port in a mid-axillary line in approximately the 6^{th} or 7^{th} intercostal space of the patient, wherein the second left port and third left port are positioned so that an instrument can access the transverse sinus, behind the superior vena cava and immediately cephalad to the right atrial appendage;
sealing at least partially the first left port, the second left port and the third left port;
opening the pericardium posterior to the phrenic nerve;
inserting the sensing pen through the most cephalad port;
obtaining a left baseline electrogram in the pulmonary veins with the sensing pen;
dividing the ligament of Marshall posterior;
introducing the lighted dissector having a tip into the most caudad port;
directing the lighted dissector around the pulmonary veins with the tip positioned at the point of the divided ligament of Marshall;
introducing a closed bipolar clamp having a jaw and a posterior jaw through the most-caudad port site;
positioning the posterior jaw behind the pulmonary vein and the clamp is closed well up on the pulmonary vein antrum;
firing the bipolar clamp 3 times changing the position of the clamp each time;
removing the lighted dissector;
obtaining a second left electrogram in the pulmonary veins wherein electrical silence indicates entrance block so that no atrial electrical activity is transmitted into the veins;
introducing the linear bipolar radiofrequency device through the most caudad port site;
forming a third linear ablation line that connects the right superior pulmonary vein over to the left superior pulmonary vein as far posteriorly as can be done in a transverse sinus;
forming a forth linear ablation line from the left fibrous trigone to the right superior pulmonary vein;
Wherein the first linear ablation line, the second linear ablation line, the third linear ablation line, and the fourth linear ablation line form an inverted triangle on the dome;
obtaining a triangle electrogram of the activity within the inverted triangle wherein a flat electrogram indicates no conducted atrial activity;
closing the pericardium;
withdrawing the first left port, the second left port and the third left port;
closing a first left port incision, a second left port incision and a third left port incision; and
inflating the lung.

2. The method of claim 1, further comprising the step of delivering radiofrequency energy from the sensing pen until all the areas second electrogram are negative for ganglionated plexi.

3. The method of claim 1, wherein the linear bipolar ablation device comprises a deflectable tip and a malleable shaft.

4. The method of claim 1, further comprising the step of positioning a spinal needle through the chest wall with the tip targeting the transverse sinus to determine the exact placement of the first right port, the second right port, the third right port, the first left port, the second left port or the third left port.

5. The method of claim 1, wherein the left side ports are placed more posteriorly.

6. The method of claim 1, further comprising the step of positioning Pericardial retraction sutures are brought out through the posterolateral chest wall.

7. The method of claim 1, wherein the first linear ablation line is placed as posteriorly on the dome of the atrium as possible.

8. The method of claim 1, wherein the object comprises three to five bath blankets.

9. The method of claim 1, further comprising the step of the step of preparing access to a groin area of the patient for urgent access.

10. The method of claim 1, wherein the external defibrillator pads are placed behind a right shoulder area and on a left flank area or over a sternum area and directly posterior on the back area of the patient.

11. The method of claim 1, wherein the first port is a 5 mm port, the second port is a 10 mm port and the third port is a 10 mm port.

12. The method of claim 1, wherein a tourniquet are placed around the first port, the second port and the third port.

13. The method of claim 1, further comprising the step of the step of pacing the pulmonary veins and look for exit block for the patient in sinus rhythm.

14. The method of claim 1, further comprising the step of detecting ganglionated plexi by delivering a high frequency stimulation.

15. The method of claim 1, further comprising the step of dissecting the fibroareolar connective tissue between the atrial dome and the right pulmonary artery.

16. The method of claim 1, further comprising the step of touching-up the second linear ablation line by using a bipolar pen to also deliver radiofrequency ablation lesions in a stamping fashion.

17. A method of forming atrial lesions on the dome of the atrium comprising the steps of:
positioning the patient on a table;
placing 3 to 6 blankets under a thorax area of the patient to raise the to gain access to a posterolateral thorax area;
preparing access to a lateral thorax area bilaterally and a sternum area of the patient;
placing one or more external defibrillator pads on the patient;
positioning a spinal needle through the chest wall with the tip targeting the transverse sinus to determine the exact placement of a first 5 mm right port, a second 10 mm right port or a 10 mm right third port;
inserting the first 5 mm right port in a mid-axillary line in the third intercostal space of the patient;
insufflating the patient with CO₂ to expand the field and depresses the diaphragm;
inserting a second right port in a mid-clavicular line in approximately a second intercostal space of the patient;
inserting a third right port in a mid-axillary line in approximately the 7^{th} intercostal space of the patient, wherein the second right port or third right port is positioned most cephalad and the other is positioned most caudad so that an instrument can access the transverse sinus, behind the superior vena cava and immediately cephalad to the right atrial appendage;
sealing at least partially the first right port, the right second port and the third right port;
dissecting the fibroareolar tissue between the inferior vena cava and the right inferior pulmonary vein to form an opening into the posterior pericardium;
converting a portion of the superior vena cava to an intrapericardial area;
dividing the tissue between the superior vena cava and the right pulmonary artery;
dissecting the fat pad located behind the superior vena cava and in front of the left atrial muscular dome to expose the atrium muscular dome;
removing the second right port or the third right port and insert a lighted dissector;
advancing the lighted dissector between the right inferior pulmonary vein and the inferior vena cava into the posterior pericardium;
dissecting through the space between the right pulmonary artery and the right superior pulmonary vein with the lighted dissector;
removing the lighted dissector;
inserting a sensing pen through the most cephalad port;
obtaining a right baseline electrogram on multiple sites on both the superior and inferior pulmonary veins with the sensing pen;
introducing a closed bipolar clamp comprising a jaw and a posterior jaw a through the most caudad port site;
positioning the posterior jaw of the clamp behind the right-sided pulmonary veins until the jaw and the posterior jaw are well-up on the antrum of the pulmonary vein and well-away from the bifurcation of the pulmonary veins;
producing an ablation line on the right pulmonary vein antrum by firing the clamp 2-6 times;
obtaining a second right electrogram on multiple sites on both the superior and inferior pulmonary veins with the sensing pen, wherein the absence of transmitted electrical activity from the atrium indicates acute entrance block has been obtained;
introducing a linear bipolar radiofrequency device through the most caudad port;
positioning the linear bipolar ablation device behind the superior vena cava;
forming a first linear ablation line from the right superior pulmonary vein across the dome of the left atrium pointing towards the left superior pulmonary vein;
positioning the linear bipolar ablation device at the junction of the noncoronary cusp and the left coronary cusp of the aorta;
forming a second linear ablation line from the fibrous trigone obliquely on the dome to connect the left fibrous trigone to the transverse ablation line across the dome of the atrium;
closing the pericardium;
withdrawing the first 5 mm right port, the second 10 mm right port and the third 10 mm right port;
closing a first right port incision, a second right port incision and a third right port incision;
inflating the lung;
positioning a first 5 mm left port in a mid-axillary line in the third intercostal space of the patient;
insufflating the patient with CO₂ to expand the field and depresses the diaphragm;
positioning a second 10 mm left port in a mid-clavicular line in approximately a second intercostal space of the patient;
positioning a third 10 mm left port in a mid-axillary line in approximately the 6^{th} or 7^{th} intercostal space of the patient, wherein the second 10 mm left port and third 10 mm left port are positioned so that an instrument can access the transverse sinus, behind the superior vena cava and immediately cephalad to the right atrial appendage;
sealing at least partially the first left port, the second left port and the third left port;
opening the pericardium posterior to the phrenic nerve;
inserting the sensing pen through the most cephalad port;
obtaining a left baseline electrogram in the pulmonary veins with the sensing pen;
dividing the ligament of Marshall posterior;
introducing the lighted dissector having a tip into the most caudad port;
directing the lighted dissector around the pulmonary veins with the tip positioned at the point of the divided ligament of Marshall;
introducing a closed bipolar clamp having a jaw and a posterior jaw through the most-caudad port site;
positioning the posterior jaw behind the pulmonary vein and the clamp is closed well up on the pulmonary vein antrum;
firing the bipolar clamp 3 times changing the position of the clamp each time;
removing the lighted dissector;
obtaining a second left electrogram in the pulmonary veins wherein electrical silence indicates entrance block so that no atrial electrical activity is transmitted into the veins;
introducing the linear bipolar radiofrequency device through the most caudad port site;
forming a third linear ablation line that connects the right superior pulmonary vein over to the left superior pulmonary vein as far posteriorly as can be done in a transverse sinus;
forming a forth linear ablation line from the left fibrous trigone to the right superior pulmonary vein;
Wherein the first linear ablation line, the second linear ablation line, the third linear ablation line, and the fourth linear ablation line form an inverted triangle on the dome;
obtaining a triangle electrogram of the activity within the inverted triangle wherein a flat electrogram indicates no conducted atrial activity;
closing the pericardium;
withdrawing the first 5 mm left port, the second 10 mm left port and the third 10 mm left port;
closing a first 5 mm left port incision, a second 10 mm left port incision and a third 10 mm left port incision; and
inflating the lung.
